(19) United States
(12) Patent Application Publication
(10) Pub. No.: US 2025/0232885 A1
(43) Pub. Date: Jul. 17, 2025

(54) MACHINE LEARNING-BASED DISEASE TRANSMISSION PREDICTIONS AND INTERVENTIONS

(71) Applicant: MatrixCare, Inc., Bloomington, MN (US)

(72) Inventor: Denver Michael WEST, Republic, MO (US)

(21) Appl. No.: 19/020,699

(22) Filed: Jan. 14, 2025

Related U.S. Application Data
(60) Provisional application No. 63/621,694, filed on Jan. 17, 2024.

(51) Int. Cl. G16H 50/80; G16H 40/20; G16H 50/30

(52) U.S. Cl. CPC G16H 50/80; G16H 40/20; G16H 50/30

(57) ABSTRACT

Techniques for machine learning-based transmission prediction are provided. A set of characteristics for a healthcare provider is accessed. A probability of transmission, with respect to a disease, is generated using a machine learning model corresponding to the disease based on the set of characteristics and a community comprising a plurality of individuals. A current configuration of a healthcare facility in the community is determined with respect to the disease. An updated configuration for the healthcare facility, with respect to the disease, is generated based on the probability of transmission.

### CROSS-REFERENCE TO RELATED APPLICATIONS

This application claims priority to U.S. Provisional Patent Application No. 63/621,694 filed Jan. 17, 2024, the entire content of which is incorporated herein by reference in its entirety.

### INTRODUCTION

Aspects of the present disclosure relate to machine learning. More specifically, aspects of the present disclosure relate to training and using machine learning to generate predictions and interventions for disease transmission.

In countless locales and communities, a wide assortment of healthcare facilities exist, often providing services ranging from general healthcare to highly specialized care. These healthcare facilities are generally staffed by a variety of care providers, such as dentists, chiropractors, clinical psychologists, nurse practitioners, registered nurses, midwives, social workers, physician assistants, doctors, and the like. In many cases, all (or substantially all) of the care providers in a given facility are from or reside in the local community. However, in many other cases, some (or a substantial portion) of the care providers are not local to the community. For example, travel nurses generally work in temporary nursing positions, and may move from facility to facility relatively frequently. Similarly, many care providers work in communities relatively remote from the communities in which they live (e.g., living in or near a large population center and working in a facility in a relatively more rural nearby community).

Working in healthcare facilities often exposes healthcare providers to a variety of communicable diseases via patients seeking care. Such individuals are also inherently exposed to such communicable diseases via their communities (e.g., when shopping, spending leisure time in the community, and the like). Current approaches to evaluate spread of communicable disease are generally limited to retrospective analysis (e.g., evaluating historical and/or current transmission rates). Given the significant complexities of disease transmission, adequate forward-facing predictions are not available today. Further, these complexities are increased substantially in the case of care providers who work outside of their local community (e.g., providers on assignment in a facility that is not located where the provider lives), rendering appropriate decision-making and prevention virtually impossible.

Improved systems and techniques to predict and intervene in disease transmission are desired.

### SUMMARY

According to one embodiment presented in this disclosure, a method is provided. The method includes: accessing a first set of characteristics for a first healthcare provider; generating a first probability of transmission, with respect to a first disease, using a first machine learning model corresponding to the first disease and based on the first set of characteristics and a first community comprising a plurality of individuals; determining a current configuration of a first healthcare facility in the first community with respect to the first disease; and generating an updated configuration for the first healthcare facility with respect to the first disease based on the first probability of transmission.

According to a second embodiment of the present disclosure, a method is provided. The method includes: accessing a first set of characteristics for a first healthcare provider; generating a first probability of transmission, with respect to a first disease, using a first machine learning model corresponding to the first disease and based on the first set of characteristics and a first community comprising a plurality of individuals; determining updated transmission data indicating whether the first healthcare provider contracted the first disease in the first community; and updating one or more parameters of the first machine learning model based on comparing the first probability of transmission and the updated transmission data.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below. The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for using machine learning to predict disease transmission, according to one embodiment of the present disclosure.

FIG. 2 depicts an example workflow for generating transmission predictions using machine learning, according to one embodiment of the present disclosure.

FIG. 3 depicts an example workflow for generating transmission predictions and updated configurations for healthcare facilities, according to one embodiment of the present disclosure.

FIG. 4 depicts an example workflow for generating transmission predictions and refining machine learning models based on subsequent transmission feedback, according to one embodiment of the present disclosure.

FIG. 5 depicts an example workflow for training machine learning models to predict disease transmission, according to one embodiment of the present disclosure.

FIG. 6 is a flow diagram depicting an example method for predicting transmission using machine learning, according to one embodiment of the present disclosure.

FIG. 7 is a flow diagram depicting an example method for determining relevant characteristics data for predicting disease transmission, according to one embodiment of the present disclosure.

FIG. 8 is a flow diagram depicting an example method for generating staffing, supply, and/or protocol modifications based on disease predictions using machine learning, according to one embodiment of the present disclosure.

FIG. 9 is a flow diagram depicting an example method for generating updated configurations based on disease predictions using machine learning, according to one embodiment of the present disclosure.

FIG. 10 is a flow diagram depicting an example method for selecting alternative providers based on disease transmission using machine learning, according to one embodiment of the present disclosure.

FIG. 11 is a flow diagram depicting an example method for refining machine learning models based on disease transmission feedback, according to one embodiment of the present disclosure.

FIG. 12 is a flow diagram depicting an example method for training machine learning models to predict disease transmission, according to one embodiment of the present disclosure.

FIG. 13 is a flow diagram depicting an example method for using machine learning to predict transmission, according to one embodiment of the present disclosure.

FIG. 14 is a flow diagram depicting an example method for training machine learning models to predict transmission, according to one embodiment of the present disclosure.

FIG. 15 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved transmission prediction and facility configuration using machine learning.

In some embodiments, a variety of relevant information related to communities, facilities, individual care providers, and disease transmission may be used to train one or more machine learning models to predict future transmission. For example, the machine learning models may be used to predict the probability that a given individual will contract one or more diseases if the individual is sent to and/or lives in a given community and/or works in a given facility. In some embodiments, the machine learning model(s) may generate predictions based on data such as (without limitation) the characteristics of the community itself (e.g., population density, transmission rates, vaccination rates, and the like), the vaccination status of the individual, prevention protocols used by the individual, facility, and/or community, and the like.

For example, in some embodiments, when a healthcare provider is considering moving to or otherwise entering a community to provide healthcare services (either temporarily or indefinitely), a transmission prediction system may be used to predict transmission probabilities for the provider. This can allow the provider to make more informed decisions, as well as to implement various prophylactic or preventative steps as appropriate. In some embodiments, a variety of potential modifications or actions can be similarly evaluated using machine learning in order to identify which configuration(s) or action(s) are most appropriate in view of the need(s) of the community and the potential for disease transmission.

Advantageously, by using trained machine learning models to predict transmission, the transmission prediction system is able to substantially improve healthcare services. For example, when accurate transmission predictions are generated using embodiments of the present disclosure, a wide variety of previously intractable tasks become solvable. As one example, staffing needs can be readily resolved. By assigning or allocating staff based at least in part on probable disease transmission, the transmission prediction system can prevent shortfalls caused by unexpected illness. Similarly, using transmission predictions, supply management is improved as the risk of over (or under) supplying a given facility or community is considerably lowered.

As another example, the provided healthcare can itself be improved, while minimizing disruption of transmission. For example, intelligent preventative decisions can be made that rely on accurate and objective transmission predictions, rather than simple heuristics or efforts implemented out of an abundance of caution. This can allow users to reserve more onerous or expensive preventative techniques only where such approaches are warranted by high transmission probabilities (determined based on objective machine learning). This reduces resource waste and reduces risk and harm caused by disease transmission.

As yet another example, the operations of the computing systems themselves may be improved, such as by using more efficient data retrieval and evaluations, as compared to conventional solutions. For example, some conventional systems require users to manually retrieve and review relevant transmission data, which can impose substantial computational burden on the systems, such as through increased bandwidth from repeated queries and data exchanges between storage repositories, increased power consumption caused by such manual searching, increased heat generation and power consumption caused by outputting such data visually to the user (e.g., requiring that a monitor be powered on and outputting data for a long period of time so the user can manually review the data, which consumes substantial energy and generates substantial heat), and the like. Further, these conventional approaches are inherently retrospective and subjective, and rely on an inaccurate heuristic-based approach rather than objective predictions.

In contrast, embodiments of the present disclosure can use targeted and specific data retrieval processes (reducing bandwidth and duplicative exchanges). Further, by using machine learning to generate transmission predictions, the resources needed to facilitate the decision process are reduced substantially (e.g., display devices need not consume power until the evaluations are complete, and the user need only review the output briefly to make an informed decision). Embodiments of the present disclosure thereby provide substantial improvements to the operations of the computer itself.

### Example Environment for Using Machine Learning to Predict Disease Transmission

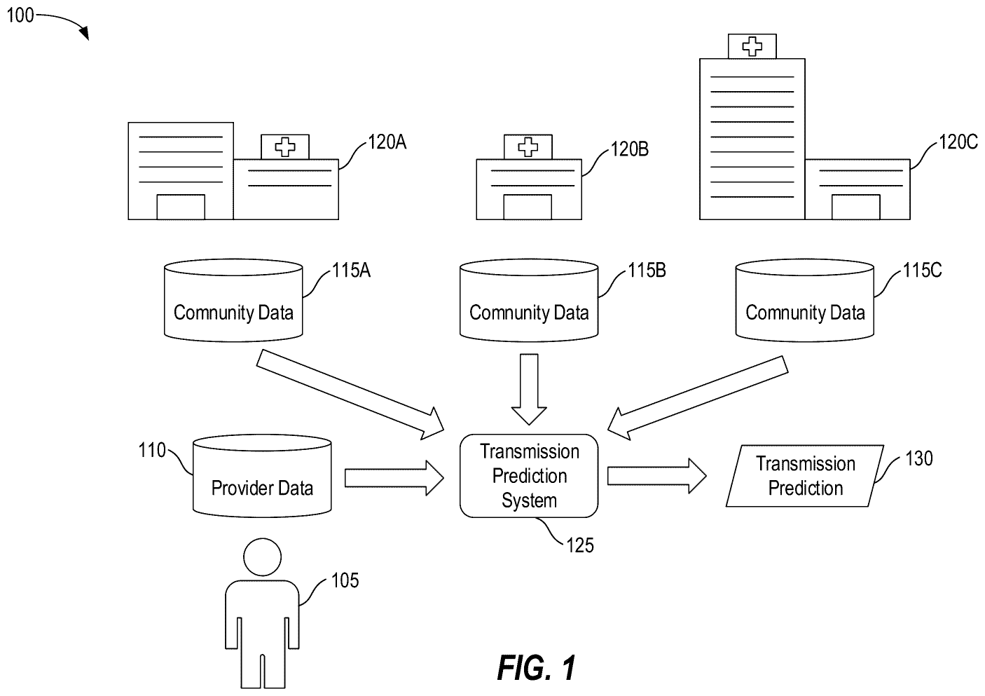

FIG. 1 depicts an example environment 100 for using machine learning to predict disease transmission, according to one embodiment of the present disclosure.

In the illustrated example, a transmission prediction system 125 accesses a variety of data, including provider data 110 and community data 115 from a variety of healthcare facilities 120 (or communities, more generally), to generate transmission predictions 130. As used herein, “accessing” data generally corresponds to receiving, requesting, retrieving, generating, collecting, obtaining, or otherwise gaining access to the data. For example, the transmission prediction system 125 may access data from local storage or memory, from one or more repositories or other systems located remotely from the transmission prediction system 125 (e.g., via one or more networks, such as the Internet), and the like.

In the illustrated example, the transmission prediction system 125 uses community data 115 specific to each healthcare facility 120 to generate transmission predictions 130. Specifically, the healthcare facility 120A has an associated set of community data 115A, the healthcare provider 120B has an associated set of community data 115B, and the healthcare facility 120C has an associated set of community data 115C. By using community-specific and/or facility-specific data, the transmission prediction system 125 may generate community-specific and/or facility-specific predictions (e.g., predicting transmission probabilities with respect to each specific community and/or healthcare facility 120), allowing for improved transmission predictions 130 and subsequent responses. Although three healthcare facilities 120 are depicted for conceptual clarity, in embodiments, the transmission prediction system 125 may evaluate data for any number and variety of facilities distributed across any number of communities.

In some embodiments, the transmission prediction system 125 trains one or more machine learning models based on the community data 115. For example, in some embodiments, the transmission prediction system 125 trains a respective machine learning model for each respective community or healthcare facility 120. That is, the transmission prediction system 125 may train a first machine learning model for the healthcare facility 120A based on the corresponding community data 115A, a second machine learning model for the healthcare facility 120B based on the corresponding community data 115B, and a third machine learning model for the healthcare facility 120C based on the corresponding community data 115C. To generate a community-specific and/or facility-specific prediction, the transmission prediction system 125 may evaluate the provider data 110 using the specific model(s) trained for the specific healthcare facility 120.

In some embodiments, the transmission prediction system 125 may train one or more machine learning models to be used across multiple healthcare facilities 120. For example, the transmission prediction system 125 may train the model(s) to receive, as part of its input, an indication of the particular healthcare facility 120 and/or other information about the facility or community for which the predictions should be generated, as discussed in more detail below.

In the illustrated example, once the machine learning model(s) are trained (as discussed in more detail below), the transmission prediction system 125 may process data such as the provider data 110 (e.g., corresponding to a healthcare provider 105) using the model(s) to generate the transmission prediction 130. For example, when the healthcare provider 105 is referred to, assigned to, or is contemplating assignment to a given healthcare facility 120, the transmission prediction system 125 may evaluate corresponding provider data 110 and/or community data 115 using the machine learning model(s) to generate the transmission predictions 130.

The provider data 110 generally includes any relevant information that can be used to evaluate disease transmission with respect to the healthcare provider 105. For example, in some embodiments, the provider data 110 may include the vaccination status of the healthcare provider 105 with respect to one or more communicable diseases, comorbidities or other conditions of the healthcare provider 105 that may impact disease transmission and/or disease severity of the disease is contracted, preventative protocols that the healthcare provider 105 uses (or is willing to use), and the like.

In some embodiments, the healthcare provider 105 (or another user, such as a manager of the healthcare provider 105) may provide the provider data 110 and request that one or more transmission predictions 130 be generated (e.g., for one or more specific healthcare facilities 120 or communities). In some embodiments, the healthcare provider 105 (or other user) may simply request transmission prediction(s) 130, and the transmission prediction system 125 may retrieve any other relevant data (e.g., from one or more data repositories) based on the provider identifier. In some embodiments, the request may indicate a specific target healthcare facility 120, and the transmission prediction system 125 may generate transmission predictions 130 for the target facility based on the provider data 110 and/or corresponding community data 115. In some embodiments, the transmission prediction system 125 may additionally evaluate alternative healthcare facilities 120 for the healthcare provider 105, and/or may evaluate alternative healthcare providers 105 for the given healthcare facility 120.

For example, the transmission prediction system 125 may evaluate a variety of providers and facilities to predict transmission predictions 130, and these predictions may be used to allocate staff to facilities in an optimal (or at least improved) manner, such as by assigning healthcare providers 105 with low transmission probabilities and/or low comorbidities to certain healthcare facilities 120 (e.g., healthcare facilities 120 with relatively high transmission rates and/or low vaccination rates), while assigning healthcare providers 105 with relatively higher transmission probabilities and/or worse comorbidities to certain other healthcare facilities 120 (e.g., facilities with relatively lower transmission rates and/or higher vaccination rates).

In an embodiment, the transmission prediction 130 can generally include a wide variety of information in response to the request. In some embodiments, the transmission prediction 130 includes one or more prediction(s) generated for one or more healthcare facilities 120. For example, the transmission prediction 130 may indicate the predicted probabilities of disease transmission for one or more diseases if the healthcare provider 105 is assigned to or works at one or more of the healthcare facilities 120. In some embodiments, the transmission prediction system 125 uses disease-specific machine learning models, where each model is trained based on disease transmission for a specific disease. In some embodiments, the transmission prediction system 125 uses disease-agnostic models, where a specific disease may be indicated as part of the input to the model in order to generate transmission predictions 130.

In some embodiments, the transmission prediction 130 can similarly include predictions related to the potential disease severity if the healthcare provider 105 contracts one or more diseases (e.g., based on comorbidities of the healthcare provider 105 and/or based on community data 115).

In some embodiments, as discussed below in more detail, the transmission prediction 130 indicates one or more changes or modifications that, if implemented by a given healthcare facility 120 and/or by the healthcare provider 105, would make that healthcare facility 120 a better fit for the healthcare provider 105 (e.g., due to reduced probability of transmission, or reduced predicted severity of the disease).

In some embodiments, the transmission prediction system 125 outputs the transmission prediction 130 to a user, such as to the healthcare provider 105 and/or to a manager who is making staffing decisions. This allows the user to determine whether to implement an assignment (e.g., whether to assign the healthcare provider 105 to a given healthcare facility 120 and/or whether to accept such an assignment). In some embodiments, the transmission prediction system 125 may itself make facility configuration decisions such as staffing and supply decisions (e.g., by comparing the transmission predictions 130 to various criteria or rules, such as maximum threshold probabilities of transmission). In these ways, as discussed above, the transmission prediction system 125 can substantially improve healthcare service provisioning, reduce resource waste, and reduce computational expense of the disease tracking process.

### Example Workflow for Generating Transmission Predictions Using Machine Learning

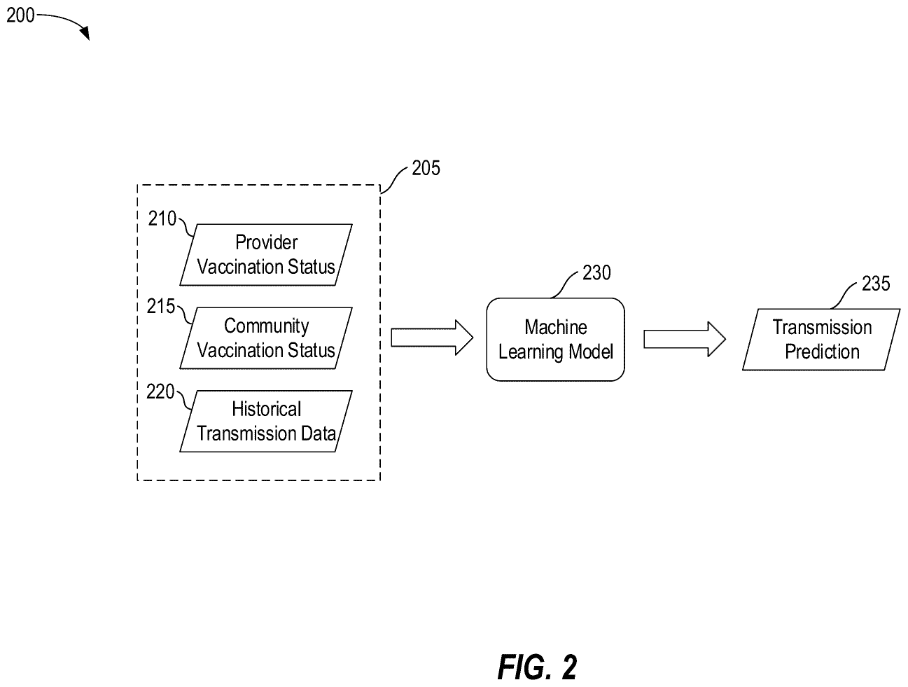

FIG. 2 depicts an example workflow 200 for generating transmission predictions using machine learning, according to one embodiment of the present disclosure. In some embodiments, the workflow 200 is performed by a transmission prediction system, such as the transmission prediction system 125 of FIG. 1.

In the illustrated example, input data 205 is accessed by a machine learning model 230 to generate one or more transmission predictions 235. In some embodiments, the input data 205 corresponds to the provider data 110 and/or community data 115 of FIG. 1. As discussed above, the input data 205 generally includes any relevant information that may be used, by the machine learning model 230, to generate the transmission prediction(s) 235. For example, in the illustrated workflow 200, the input data 205 includes information such as the provider vaccination status 210, community vaccination status 215, and historical transmission data 220.

As discussed above, the provider vaccination status 210 generally corresponds to or indicates whether a given healthcare provider (e.g., the healthcare provider 105 of FIG. 1) is vaccinated against one or more communicable diseases. In some embodiments, the provider vaccination status 210 may comprise information specific to the transmission predictions 235 that are being generated. For example, if the transmission predictions 235 are being generated to predict whether the provider will contract the flu, the provider vaccination status 210 may indicate whether the provider has been vaccinated for the flu.

In some embodiments, the provider vaccination status 210 is a binary value (e.g., indicating that the provider is vaccinated or unvaccinated). In some embodiments, the provider vaccination status 210 comprises more granular data, such as identifying the specific vaccine (if any) that the provider received. For example, the provider vaccination status 210 may specifically indicate which vaccine(s) were received if vaccines from multiple manufacturers are available, vaccines targeting different strains of the disease are available, different types of vaccines are available, and/or vaccines targeting different combinations of diseases are available, and the like. As another example, in some embodiments, the provider vaccination status 210 may indicate the timing of the vaccination and/or whether any boosters (if available) were received. For example, the provider vaccination status 210 may indicate how much time has elapsed since the vaccine was received (in the case of vaccines that provide more temporary protection, such as a tetanus, diphtheria, and pertussis (Tdap) vaccine) and/or whether the provider is due for a booster. Similarly, in some embodiments, if the vaccine comprises a course of doses (e.g., multiple doses over a defined period of time), the provider vaccination status 210 may indicate whether the provider has completed the course (or how far along the course they are).

As discussed above, the community vaccination status 215 may generally comprise similar information to the provider vaccination status 210, but on the community-level. That is, the community vaccination status 215 may correspond to information about the vaccination rates or status with respect to one or more diseases across a community. For example, the community vaccination status 215 may indicate the percentage of individuals in the community who are vaccinated, the proportions of each specific vaccine alternative that individuals received, the average amount of time that has elapsed since each individual was vaccinated, and the like.

As discussed above, a community (also referred to as a locale) generally corresponds to a collection of individuals that share a physical proximity, such that communicable diseases may transfer among the individuals in the community. In some embodiments, the community can be defined using any level of granularity. For example, a community may correspond to the individuals that live and/or work in a defined geographic region, such as in a specific healthcare facility, in a specific town (or portion thereof), city (or portion thereof), county (or portion thereof), state (or portion thereof), country (or portion thereof), and the like.

In some embodiments, the community vaccination status 215 includes data for a single community (e.g., the community where the healthcare facility 120A operates, corresponding to community data 115A of FIG. 1). For example, if the transmission prediction 235 is being generated to evaluate disease transmission if the provider is assigned to or moves to a specific community, the community vaccination status 215 may include information for this community. In some embodiments, the community vaccination status 215 may include information for multiple communities. For example, if the transmission prediction 235 is being generated to compare probable disease transmission for multiple alternative communities, the community vaccination status 215 may include information for these alternatives. As another example, if the transmission prediction 235 is being generated to evaluate disease transmission if the provider lives in one community and commutes to another community to work, the community vaccination status 215 may include information for these communities.

In some embodiments, the historical transmission data 220 includes data related to transmission of one or more communicable diseases in one or more communities. For example, the historical transmission data 220 may indicate the most-recent transmission rate (e.g., the number of new disease contractions in the last week, the number of new contractions as a proportion of the total population of the community, and the like). In some embodiments, the historical transmission data 220 may indicate historical trends in the transmission rate, such as the transmission rate for several points or windows (e.g., each week for the last month).

In the illustrated workflow 200, the machine learning model 230 processes the input data 205 to generate the transmission predictions 235. In some embodiments, as discussed above, the machine learning model 230 may process the input data 205 in response to a user request (e.g., when a user is considering moving to or working in a specific community, or is trying to decide where to move), in response to a manager request (e.g., when a manager is determining whether to assign the provider to a community), and the like.

Although not depicted in the illustrated example, in some embodiments, the input data 205 may undergo various preprocessing and/or feature extraction prior to being processed by the machine learning model 230. For example, the machine learning model 230 (or another component) may extract relevant or salient features, vectorize the data, and the like.

As discussed above, each transmission prediction 235 generally comprises a prediction relating to what will happen, to the healthcare provider, if the provider is assigned to or otherwise physically enters the community (e.g., to work and/or live in the community). For example, the transmission predictions 235 may indicate a categorical prediction as to whether the provider will contract one or more disease(s). In some embodiments, the transmission predictions 235 may include one or more predicted probabilities about the outcome (e.g., the probability that the provider will contract the disease).

In some embodiments, the transmission predictions 235 may include predictions about the predicted severity of the disease(s), if the provider contracts the disease(s), such as whether the provider will require hospitalization or other intervention, how long it will take for the provider to recover, and the like. In some embodiments, the transmission predictions 235 may include timeline-related predictions, such as how much time will pass before the provider contracts the disease.

Generally, the particular contents of the transmission predictions 235 may vary depending on the particular implementation. In some embodiments, the transmission predictions 235 may additionally or alternatively include one or more aggregate predictions. For example, in addition to or instead of indicating the probability that the provider will contract the disease as well as the predicted severity if the provider does contract the disease, the transmission predictions 235 may generate an aggregated score (e.g., a value between zero and one) indicating the overall risk, based on combining the probability of contraction and the probable severity of contraction.

In some embodiments, each of the transmission predictions 235 may be generated by a corresponding machine learning model 230. That is, the machine learning model 230 may be trained to predict a specific type of outcome (e.g., contraction recovery) for a specific disease, while a second model may be trained to predict another outcome (e.g., predicted severity). In some embodiments, therefore multiple models may be used to generate a set of transmission predictions 235. In other embodiments, one or more of the models may be trained to predict multiple predicted outcomes.

In some embodiments, the transmission predictions 235 may be community-specific. That is, each transmission predictions 235 may indicate a prediction if the provider is assigned to or moves to a specific facility or community. In some embodiments, the machine learning model 230 is similarly community-specific. For example, there may be a separate machine learning model 230 trained for each respective community (e.g., where each model is trained based on data from the corresponding community). This may allow the models to specialize with respect to any uniqueness of each community that may otherwise be difficult or impossible to adequately capture in training data (e.g., whether individuals in the community tend to cluster closely together or stand further apart, whether individuals in the community tend to associate in large groups or small settings, whether individuals in the community tend to spend more time indoors or outdoors, and the like).

In some embodiments, the machine learning model 230 may be community-agnostic. For example, the machine learning model 230 may be trained using data from multiple communities. In some embodiments, to generate community-specific predictions using a community-agnostic model, the machine learning model 230 may receive, as input, an indication of a specific community (e.g., a unique identifier of the community, and/or implicitly indicated in the community vaccination status 215 and/or historical transmission data 220).

As discussed above, in some embodiments, the transmission predictions 235 may be used to facilitate a variety of decisions and actions. For example, in some embodiments, the transmission predictions 235 may be provided to a user (e.g., a healthcare provider or manager) to determine whether the provider should move to or begin working in a given facility or community. In some embodiments, the transmission predictions 235 may be evaluated automatically (e.g., by a transmission prediction system) to generate a suggestion or recommendation (e.g., by comparing the transmission predictions 235 to one or more criteria).

In some embodiments, the transmission predictions 235 may be used to generate suggestions or recommendations, such as to identify another community that would be a better fit for the provider (e.g., that will provide better transmission predictions 235), or to indicate changes that the provider, community, and/or healthcare facility could make to improve the transmission predictions 235, as discussed below in more detail.

### Example Workflow for Generating Transmission Predictions and Updated Configurations for Healthcare Facilities

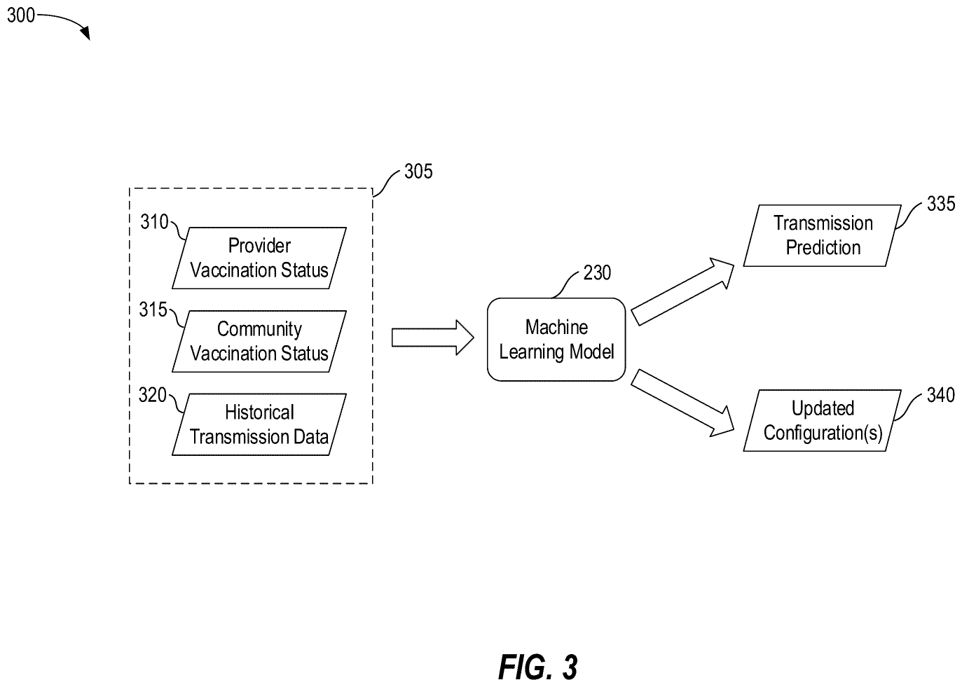

FIG. 3 depicts an example workflow 300 for generating transmission predictions and updated configurations for healthcare facilities, according to one embodiment of the present disclosure. In some embodiments, the workflow 300 is performed by a transmission prediction system, such as the transmission prediction system 125 of FIG. 1.

In the illustrated example, input data 305 is accessed by a machine learning model 230 to generate one or more transmission predictions 335, as well as updated configuration(s) 340. In some embodiments, the input data 305 corresponds to the provider data 110 and/or community data 115 of FIG. 1, and/or the input data 205 of FIG. 2. As discussed above, the input data 305 generally includes any relevant information that may be used, by the machine learning model 230, to generate the transmission predictions 335 and updated configurations 340. For example, in the illustrated workflow 300, the input data 305 includes information related to the provider vaccination status 310, the community vaccination status 315, and the historical transmission data 320.

As discussed above, the provider vaccination status 210 generally corresponds to or indicates information about the vaccination status of a given healthcare provider (e.g., the healthcare provider 105 of FIG. 1) with respect to one or more communicable diseases (e.g., a binary indication of vaccination status, a time indication of how much time has passed since vaccination, the type(s) of vaccination received, and the like. The community vaccination status 315 generally includes similar information on a community-wide basis, such as the proportion of the community that is vaccinated. As discussed above, the historical transmission data 320 generally includes data related to transmission of the one or more communicable diseases in one or more communities (e.g., indicating the most-recent transmission rate and/or historical trends in the transmission rate).

In the illustrated workflow 300, the machine learning model 230 processes the input data 305 to generate the transmission predictions 335. Although not depicted in the illustrated example, in some embodiments, the input data 305 may undergo various preprocessing and/or feature extraction prior to being processed by the machine learning model 230. For example, the machine learning model 230 (or another component) may extract relevant or salient features, vectorize the data, and the like.

As discussed above, each transmission prediction 335 generally comprises a prediction relating to disease transmission if the indicated healthcare provider is assigned to, works in, and/or moves to a given target community or healthcare facility. For example, the transmission predictions 335 may indicate a categorical prediction as to whether the provider will contract the disease(s), the predicted amount of time that will elapse before disease contraction, the predicted severity of the disease, the predicted duration during which the provider will be contagious and/or ill (e.g., unable to work) due to the disease, and the like. Generally, the particular contents of the transmission predictions 335 may vary depending on the particular implementation.

As discussed above, in some embodiments, each of the transmission predictions 335 may be generated by a corresponding machine learning model 230 (e.g., the machine learning model 230 may be trained to predict a specific type of outcome and/or outcomes for a specific disease). That is, multiple models may be used to generate a set of transmission predictions 335.

In some embodiments, as discussed above, the transmission predictions 335 may be disease-specific and/or community-specific. That is, each transmission prediction 335 may indicate a prediction with respect to a given disease if the provider works in a given facility and/or moves to a given community. As discussed above, in some embodiments, the transmission predictions 335 may be used to facilitate staffing and other decisions. For example, in some embodiments, the transmission predictions 335 may be provided to a user (e.g., the healthcare provider, or a manager) to determine whether the assignment should be made or accepted. In some embodiments, the transmission predictions 335 may be evaluated automatically (e.g., by a transmission prediction system) to generate a suggestion or recommendation (e.g., by comparing the transmission predictions 335 to one or more criteria).

In the illustrated example, the machine learning model 230 is also used to generate one or more updated configurations 340. The updated configurations 340 generally comprise one or more suggestions, instructions, or recommendations with respect to how various protocols or other mutable characteristics of the provider and/or healthcare facility can be modified in order to improve the transmission predictions 335, as discussed in more detail below.

In some embodiments, as discussed above, the transmission prediction system may evaluate multiple alternative healthcare providers (with respect to a given community) and/or evaluate multiple alternative communities or facilities (with respect to a given healthcare provider). In some embodiments, rather than evaluating multiple providers and/or communities initially, the transmission prediction system may first evaluate the actual facility target (e.g., the healthcare facility that is under consideration for the provider) and/or the actual provider target (e.g., the healthcare provider that is being considered to work in a facility) to generate transmission predictions 335 for this target assignment. In some embodiments, the transmission prediction system only proceeds to evaluate alternative providers and/or communities (or facilities) if the transmission predictions 335 indicate that the initial target assignment is not a good fit.

For example, the transmission prediction system may use the machine learning model(s) 230 to generate transmission predictions 335 for the target assignment (e.g., for a specific healthcare provider being assigned to a specific community and/or healthcare facility). If the transmission predictions 335 fail to meet defined criteria (e.g., the probability of the provider contracting a disease is high, or the predicted severity of such a disease is high for the provider), the transmission prediction system may then determine to use the machine learning model(s) 230 to generate new transmission predictions for one or more alternative assignments (e.g., for one or more different healthcare providers being assigned to the initial community, and/or for the initial provider being assigned to one or more other communities). This can substantially reduce the computational expense, as the model(s) need only be used to process data once (for the target combination) unless the target is ill-suited. In that case, the models may be used again (incurring additional computational expense) to process alternatives. By using such an iterative approach, the transmission prediction system may reduce the computational expense of processing input data to evaluate transmission possibilities.

In some embodiments, the updated configuration(s) 340 indicate mutable characteristics or attributes of the provider and/or facility that, if changed, may improve the transmission predictions. For example, as discussed above, the updated configurations 340 may indicate or suggest actions, such as assigning additional staff, assigning a different provider to the target facility, assigning the target provider to a different facility, and the like. In some embodiments, the updated configurations 340 may indicate new or changed protocols, such as transmission prevention measures. For example, the transmission prediction system may suggest that the provider and/or other individuals in the facility should begin washing their hands more often, wearing masks, using additional sterilization or cleaning techniques, and the like. In some embodiments, the updated configurations 340 may indicate new or changed transmission detection procedures, such as increased screening or testing requirements (e.g., testing weekly instead of in response to symptom onset).

In some embodiments, to generate the updated configurations 340, the transmission prediction system may compare the characteristics of the target facility (or provider) to the characteristics of one or more other facilities (or providers). For example, in response to determining that an alternative healthcare provider is better suited for a given facility (e.g., with better transmission predictions 335), the transmission prediction system may identify difference(s) between this alternative provider and the original target provider (e.g., determining that the other provider has expressed willingness or planning to socially distance, and suggesting that the target provider consider such solutions). As another example, in response to determining that an alternative facility is better suited for a given healthcare provider (e.g., with better transmission predictions 335), the transmission prediction system may identify difference(s) between this target facility and the original target facility (e.g., determining that the other facility has a lower patient-to-staff ratio, or placed hand sanitizer in every room in the facility).

In some embodiments, once such differences are identified, they may be indicated as updated configurations 340, which may be output to a user for review (e.g., to an administrator or manager at the facility, to the healthcare provider, and the like). This can substantially improve the efficacy and quality of healthcare provided, while ensuring more efficient and targeted use of resources across systems.

### Example Workflow for Generating Transmission Predictions and Refining Machine Learning Models Based on Subsequent Transmission Feedback

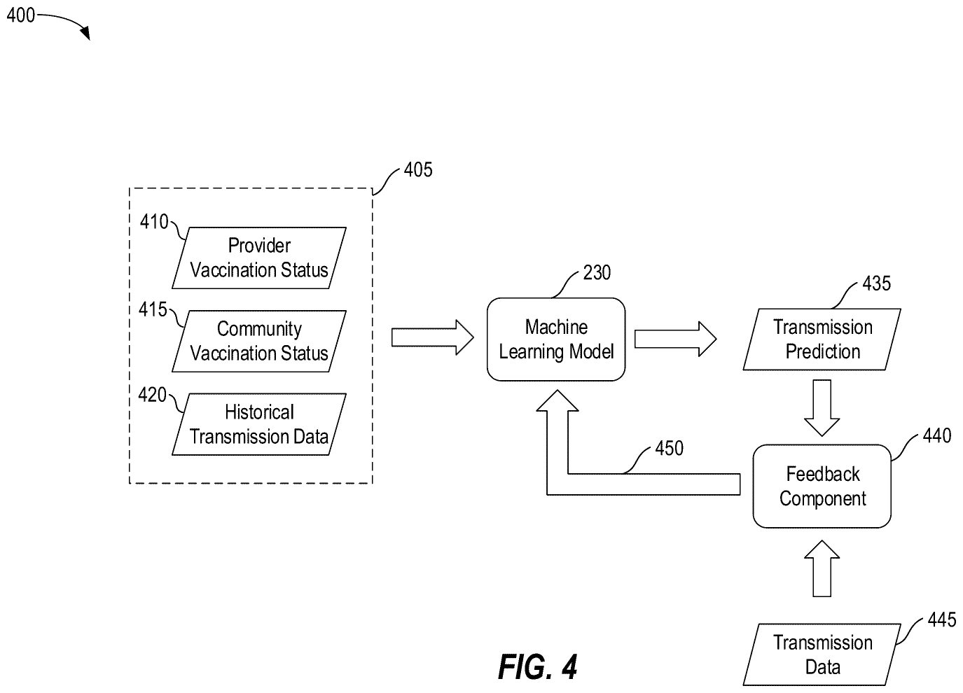

FIG. 4 depicts an example workflow 400 for generating transmission predictions and refining machine learning models based on subsequent transmission feedback, according to one embodiment of the present disclosure. In some embodiments, the workflow 400 is performed by a transmission prediction system, such as the transmission prediction system 125 of FIG. 1.

In the illustrated example, input data 405 is accessed by a machine learning model 230 to generate one or more transmission predictions 435. In some embodiments, the input data 405 corresponds to the provider data 110 and/or community data 115 of FIG. 1, the input data 205 of FIG. 2, and/or the input data 305 of FIG. 3. As discussed above, the input data 405 generally includes any relevant information that may be used, by the machine learning model 230, to generate the transmission predictions(s) 435. For example, in the illustrated workflow 400, the input data 405 includes provider vaccination status 410, community vaccination status 415, and historical transmission data 420.

As discussed above, the provider vaccination status 210 generally corresponds to or indicates information about the vaccination status of a given healthcare provider (e.g., the healthcare provider 105 of FIG. 1) with respect to one or more communicable diseases (e.g., a binary indication of vaccination status, a time indication of how much time has passed since vaccination, the type(s) of vaccination received, and the like. The community vaccination status 315 generally includes similar information on a community-wide basis, such as the proportion of the community that is vaccinated. As discussed above, the historical transmission data 320 generally includes data related to transmission of the one or more communicable diseases in one or more communities (e.g., indicating the most-recent transmission rate and/or historical trends in the transmission rate).

In the illustrated workflow 400, the machine learning model 230 processes the input data 405 to generate the transmission predictions 435. Although not depicted in the illustrated example, in some embodiments, the input data 405 may undergo various preprocessing and/or feature extraction prior to being processed by the machine learning model 230. For example, the machine learning model 230 (or another component) may extract relevant or salient features, vectorize the data, and the like.

As discussed above, each transmission prediction 435 generally comprises a prediction relating to disease transmission if the indicated healthcare provider is assigned to, works in, and/or moves to a given target community or healthcare facility. For example, the transmission predictions 435 may indicate a categorical prediction as to whether the provider will contract the disease(s), the predicted amount of time that will elapse before disease contraction, the predicted severity of the disease, the predicted duration during which the provider will be contagious and/or ill (e.g., unable to work) due to the disease, and the like. Generally, the particular contents of the transmission predictions 435 may vary depending on the particular implementation.

In the illustrated workflow 400, after an assignment accepted or declined (e.g., after a provider based moves to a specific community and/or begins working in a specific facility), a feedback component 440 may monitor the provider's status to determine how well the transmission predictions 435 align with real transmission data 445. For example, the feedback component 440 may evaluate the transmission data 445 at the target facility and/or for the specific provider (e.g., to determine whether the provider has contracted the disease), and compare these actual outcomes to the transmission prediction(s) 435.

In some embodiments, the feedback component 440 determines the transmission data 445 after one or more defined criteria are satisfied, such as a defined time period, the occurrence of a defined event, and the like. For example, in some embodiments, the feedback component 440 may determine whether the provider has been diagnosed with a communicable disease. If so, the feedback component 440 may determine the transmission data 445. (e.g., which disease(s) the provider contracted, how much time elapsed before the disease was contracted, the severity of the disease with respect to the provider, and the like). Generally, the particular transmission data 445 evaluated may depend on the particular criteria and/or disease.

In the illustrated example, based on comparing the transmission predictions 435 and the transmission data 445, the feedback component 440 can update the machine learning model(s) 230, as illustrated by arrow 450. Generally, the specific techniques used to update the machine learning models 230 may vary depending on the particular implementation and architecture. For example, if the machine learning model 230 is a neural network architecture, the feedback component 440 may generate a loss based on the transmission predictions 435 and actual transmission data 445, and update the model based on backpropagating the loss through the network. Generally, the particular loss formulation may vary depending on the particular implementation. For example, for categorical or probability predictions (e.g., predicting the likelihood that the provider will contract the disease), the feedback component 440 may use a cross-entropy loss formulation. As another example, for regression predictions (e.g., predicting the time that will elapse until the provider recovers from the disease), the feedback component 440 may use a mean squared error loss formulation.

In this way, the machine learning model 230 can be updated during inferencing, enabling the transmission prediction system to continue to generate highly accurate predictions using continuously updated models. In some embodiments, the transmission prediction system may update the machine learning model 230 periodically (e.g., the losses may be aggregated or stored until the periodic updating time arrives, at which point the model may be updated based on all generated losses). This may allow the transmission prediction system to update the models during off-peak times, such as overnight, when computational load on the system is minimized. In this way, the transmission prediction system can more efficiently provide continuous training of the models without harming other ongoing workloads (such as inferencing itself), improving the functionality and operations of the transmission prediction system and the model.

### Example Workflow for Training Machine Learning Models to Predict Disease Transmission

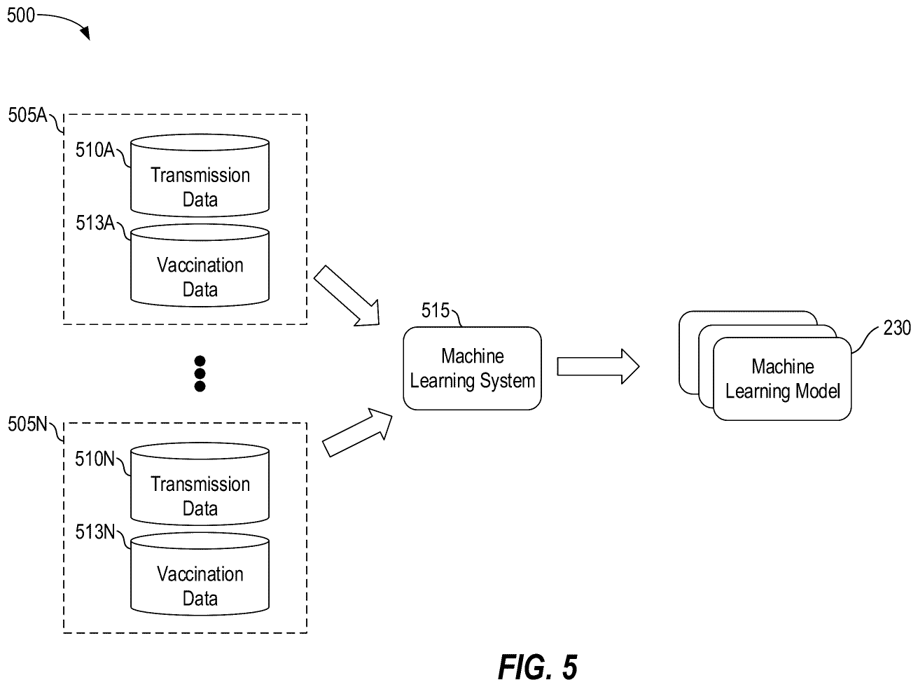

FIG. 5 depicts an example workflow 500 for training machine learning models to predict disease transmission, according to one embodiment of the present disclosure. In some embodiments, the workflow 500 is performed by a transmission prediction system, such as the transmission prediction system 125 of FIG. 1 (e.g., the machine learning system 515 may correspond to the transmission prediction system 125 of FIG. 1). That is, transmission prediction system may perform both model training as well as inferencing using trained models. In other embodiments, the machine learning system 515 is a separate system. That is, one system may be used to train the models, while another system is used to inference based on the trained models.

In the illustrated workflow 500, sets of training data 505A-N(collectively, training data 505) are accessed by a machine learning system 515, which trains a set of one or more machine learning models 230. In the illustrated example, two distinct sets of training data 505 are depicted. In embodiments, there may be any number and variety of sets of training data 505.

In some embodiments, each set of training data 505 corresponds to a respective community and/or healthcare facility (e.g., a healthcare facility 120 of FIG. 1). For example, the training data 505A may correspond to a first healthcare facility, while the training data 505N may correspond to a second healthcare facility. That is, the training data 505A may include data that is associated with and/or received from the first healthcare facility (e.g., based on the individuals and disease transmission in the facility and/or the local community around the facility), while the training data 505N may include similar data associated with and/or received from the second healthcare facility. By using such facility-specific (or community-specific) training data 505, the machine learning system 515 may train machine learning models 230 that can generate community-specific predictions, as discussed below in more detail.

In the illustrated example, each set of training data 505 comprises a set of transmission data 510 and a set of vaccination data 513. Specifically, the training data 505A comprises transmission data 510A and vaccination data 513A, while the training data 505N comprises transmission data 510N and vaccination data 513N. In some embodiments, the transmission data 510 generally comprises information about prior disease transmission at the corresponding healthcare facility and/or in the corresponding community. For example, the transmission data 510 may include information about transmission rates.

In some embodiments, the transmission data 510 may comprise or correspond to information from the community data 115 of FIG. 1 (e.g., the transmission rates in the community), all or a portion of the input data 205 of FIG. 2 (e.g., the historical transmission data 220), all or a portion of the input data 305 of FIG. 3 (e.g., the historical transmission data 320), all or a portion of the input data 405 of FIG. 4 (e.g., the historical transmission data 420), and/or the transmission data 445 of FIG. 4. In some embodiments, the transmission data 510 includes a set of records (also referred to as a sample and/or an exemplar), where each record comprises data about an individual (e.g., a healthcare provider) that contracted or was exposed to a communicable disease.

In some embodiments, the transmission data 510 includes information as of some specific time in the past. That is, rather than including the current or up-to-date information for each facility, the transmission data 510 may reflect the transmission data as of a prior time, such as when a healthcare provider began working in the facility. In some embodiments, the information in the transmission data 510 is used as the output, label, or target portion of each training exemplar, as discussed in more detail below.

In the illustrated example, the vaccination data 513 generally corresponds to or comprises information about vaccination status or rates of individuals in the healthcare facility and/or community. For example, the vaccination data 513 may correspond to the provider data 110 of FIG. 1 (e.g., indicating whether a given provider, who later contracted a disease, was vaccinated against the disease), all or a portion of the community data 115 (e.g., vaccination rates for the community), the provider vaccination status 210 and/or community vaccination status 215 of FIG. 2, the provider vaccination status 310 and/or community vaccination status 315 of FIG. 3, and/or the provider vaccination status 410 and/or community vaccination status 415 of FIG. 4. As discussed above, the particular information included in the vaccination data 513 may vary depending on the particular implementation.

In some embodiments, the vaccination data 513 includes a set of records, where each record comprises outcome information for an individual (e.g., a healthcare provider) in a facility. In some embodiments, there is a one-to-one mapping between the transmission data 510 and vaccination data 513 with respect to each set of training data 505. That is, each training exemplar may comprise a pair of records: one from the transmission data 510 (e.g., indicating whether a given healthcare provider contracted a disease while working at a healthcare facility), and one from the vaccination data 513 (e.g., indicating whether the same healthcare provider was vaccinated, and/or indicating other vaccination information for the community).

For example, for each record in the transmission data 510A, there may be a corresponding record in the vaccination data 513A (and vice versa), each corresponding to the same healthcare provider. Similarly, for each record in the transmission data 510N, there may be a corresponding record in the vaccination data 513N (and vice versa), each corresponding to the same provider. In some embodiments, the information in the vaccination data 513 is used as the input portion of each training exemplar, as discussed in more detail below.

In the illustrated example, the machine learning system 515 uses the sets of training data 505 to train one or more machine learning models 230. In some embodiments, as discussed above, the machine learning system 515 trains community-specific machine learning models 230. That is, for each community (or for each healthcare facility), the machine learning system 515 may train a corresponding set of one or more facility-specific (or community-specific)) models based on a corresponding set of training data 505. For example, the machine learning system 515 may use the training data 505A (from a first healthcare facility) to train a first set of machine learning models 230 for the first healthcare facility, and use the training data 505N (from a second healthcare facility) to train a second set of machine learning models 230 for the second healthcare facility.

By training each machine learning model 230 based only on data from a corresponding healthcare facility, the machine learning system 515 can create specialized models that can be used to generate transmission predictions for the corresponding facility. For example, during runtime, the machine learning system 515 may determine which healthcare facility is the prediction target, and use the corresponding set of model(s) to process the provider data. This can allow the models to learn to account for various facility-specific features that may otherwise be difficult or impossible to quantify (or even unknowable).

In some embodiments, rather than training a separate set of models for each healthcare facility, the machine learning system 515 may use the identity of each facility (or facility-specific information, such as the community transmission rates) as a separate input to the models during training and inferencing. For example, the machine learning system 515 may train a unified or global set of machine learning models 230 based on all sets of training data 505. In some embodiments, during such training, the machine learning system 515 may include an indication of the healthcare facility (e.g., a unique identifier) that corresponds to each exemplar. For example, when processing or training based on an exemplar from the training data 505A, the machine learning system 515 may use a unique identifier of the first healthcare facility (in addition to transmission data 510) as input to the model.

By training the machine learning models 230 based on data across facilities, the machine learning system 515 can create generalized models. Nevertheless, these models may still be used to generate facility-specific predictions, such as by providing the unique identifier of the target as input during inferencing (alongside other data).

In some embodiments, the machine learning system 515 may train machine learning models 230 that are disease-specific. For example, for a first disease, the machine learning system 515 may train a first machine learning model 230 (based on training data 505 corresponding to contraction or transmission of the first disease). For a second disease, the machine learning system 515 may train a second machine learning model 230. By training each machine learning model 230 based only on data relevant to a corresponding disease, the machine learning system 515 can create specialized models that can be used to generate transmission predictions for the corresponding disease. For example, during runtime, the machine learning system 515 may determine which disease(s) are of interest, and use the corresponding set of model(s) to process the data.

In some embodiments, rather than training a separate set of models for each disease condition, the machine learning system 515 may use the identity of each disease as a separate input to the models during training. For example, the machine learning system 515 may train a unified or global set of machine learning models 230 based on all sets of training data 505. In some embodiments, during such training, the machine learning system 515 may include an indication of the disease (e.g., a unique identifier) that corresponds to each exemplar. For example, when processing or training based on an exemplar corresponding contracting a first disease, the machine learning system 515 may use a unique identifier of the first disease (in addition to other data from the exemplar) as input to the model.

In some embodiments, the machine learning system 515 may train machine learning models 230 that are outcome or target-specific. For example, for a first outcome (e.g., whether or not the provider will contract the disease), the machine learning system 515 may train a first machine learning model 230 (based on training data 505 corresponding to the first outcome, such as with binary labels indicating whether a corresponding provider contracted the disease). For a second outcome (e.g., the predicted severity of the disease, if contracted)), the machine learning system 515 may train a second machine learning model 230 (based on training data 505 corresponding to the second outcome), and so on. Generally, the particular output labels may differ across each outcome/model. In some aspects, the particular input data may similarly change across outcomes. For example, to predict whether the provider will contract the disease, information such as the historical transmission rate in the community may be particularly relevant. Similarly, to predict disease severity, information relating to comorbidities of the provider may be useful. By training each machine learning model 230 based only on data relevant to a corresponding outcome, the machine learning system 515 can create specialized models that can be used to generate outcome-specific predictions for the corresponding outcomes. For example, during runtime, the machine learning system 515 may use a set of models to generate corresponding predictions for each relevant outcome (e.g., categorical and/or continuous value predictions for each outcome).

In some embodiments, rather than training a separate set of models for each outcome, the machine learning system 515 may train a model to predict multiple outcomes simultaneously, and/or may use the identity of each outcome as a separate input to the models during training. For example, the machine learning system 515 may train a unified or global set of machine learning models 230 based on all sets of training data 505. In some embodiments, during such training, the machine learning system 515 may include an indication of the relevant outcome (e.g., a unique identifier of the outcome for which the system would like to generate a prediction).

Generally, training the machine learning models 230 includes updating one or more parameters (e.g., weights) of the models based on the training data 505. For example, the parameters may be initialized (e.g., to random values), and the machine learning system 515 may use the training data 505 to iteratively learn values for the parameter(s) that enable improved predictions. The particular operations used by the machine learning system 515 to train the machine learning models 230 may vary depending on the particular implementation and architecture.

For example, if the machine learning models 230 use neural network architectures, the machine learning system 515 may process the input portion of a given training record (e.g., the vaccination data 513 and/or historical transmission rates in the transmission data 510) as input to the model to generate an output (e.g., one or more predicted outcomes, such as whether a given provider contracted the disease). In some aspects, as discussed above, a unique identifier of the community and/or healthcare facility to which each training record corresponds may also be used as input to generate the predicted outcome(s). The machine learning system 515 may then compare the generated output with the label portion of the record (e.g., whether the provider actually contracted the disease, as indicated in the transmission data 510) to generate a loss (e.g., using cross entropy, mean squared error, and the like). This loss can then be used to update the parameters of the model, such as via backpropagation.

Generally, the machine learning system 515 repeat this process for each training exemplar across one or more iterations or epochs of training. For example, each epoch may correspond to a single pass of each record through the models (where other aspects, such as training hyperparameters, may change between epochs).

In an embodiment, once the machine learning models 230 are fully trained, the machine learning system 515 may deploy them for inferencing. As discussed below in more detail, the machine learning system 515 may use a variety of termination criteria to determine when to deploy the models. For example, the machine learning system 515 may deploy the models after a defined number of iterations or epochs have been performed, after no additional training data 505 remains for processing, after the models have reached a preferred accuracy level, and the like.

As discussed below in more detail, deploying the machine learning models 230 can generally include any operations needed to prepare or provide them for inferencing. For example, the machine learning system 515 may compile or package the learned parameters and architecture, transmit the model(s) to one or more inferencing systems, deploy the model(s) for local inferencing (e.g., allocating memory space), and the like.

### Example Method for Predicting Transmission Using Machine Learning

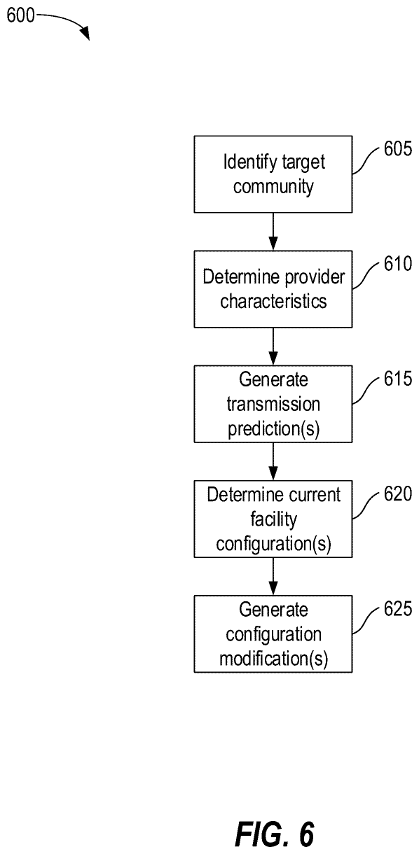

FIG. 6 is a flow diagram depicting an example method 600 for predicting transmission using machine learning, according to one embodiment of the present disclosure. In some embodiments, the method 600 is performed by a transmission prediction system, such as the transmission prediction system 125 of FIG. 1.

At block 605, the transmission prediction system identifies a target community for a given healthcare provider. For example, as discussed above, the healthcare provider (e.g., provider 105 of FIG. 1) may be considering moving to the target community and/or beginning to work in a healthcare facility (e.g., healthcare facility 120 FIG. 1) in the target community. As another example, manager or other individual in charge of staffing decisions may be considering assigning the healthcare provider to work in a healthcare facility in the target community. As discussed above, this work may correspond to a temporary work assignment (e.g., working as a travel nurse) or a potential permanent (or at least indefinite) move.

Although not depicted in the illustrated example, in some embodiments, the transmission prediction system also determines or identifies one or more characteristics of the target community (e.g., community data 115 of FIG. 1). Generally, the particular characteristics may vary depending on the particular implementation. For example, in some embodiments, the transmission prediction system may determine information related to transmission of one or more diseases in the community (e.g., historical transmission data 220 of FIG. 2, historical transmission data 320 of FIG. 3, and/or historical transmission data 420 of FIG. 4). As another example, in some embodiments, the transmission prediction system may determine information related to vaccination of individuals in the community (e.g., community vaccination status 215 of FIG. 2, community vaccination status 315 of FIG. 3, and/or community vaccination status 415 of FIG. 4).

As additional non-limiting examples, in some embodiments, the transmission prediction system may determine other characteristics of the community and/or facility that may affect disease transmission, such as population density and/or total population size in the community, staff-to-patient ratios in the facility, the total number of patients in the facility, any preventative measures or protocols in use by the community and/or facility, and the like.

At block 610, the transmission prediction system determines one or more provider characteristics (e.g., indicated in the provider data 110 of FIG. 1). Generally, the particular provider characteristics may vary depending on the particular implementation. For example, in some embodiments, the transmission prediction system may determine information related to vaccination of the healthcare provider (e.g., provider vaccination status 210 of FIG. 2, provider vaccination status 310 of FIG. 3, and/or provider vaccination status 410 of FIG. 4). As additional non-limiting examples, in some embodiments, the transmission prediction system may determine other characteristics of the provider that may affect disease transmission and/or disease severity, such as comorbidities of the provider, any preventative measures or protocols the provider plans to use and/or is willing to use, and the like.

At block 615, the transmission prediction system generates one or more transmission predictions (e.g., transmission prediction 130 of FIG. 1, transmission prediction 235 of FIG. 2, transmission prediction 335 of FIG. 3, and/or transmission prediction 435 of FIG. 4) based on the target community and/or provider characteristics. For example, as discussed above, the transmission prediction system may process the community data (e.g., vaccination rates, transmission rates, and the like) and the provider data (e.g., the provider's vaccination status) using one or more trained machine learning models (e.g., machine learning model 230 of FIGS. 2-5) to generate the transmission prediction(s).

As discussed above, the contents of the transmission predictions may vary depending on the particular implementation. For example, in some embodiments, the transmission predictions indicate the probability of transmission of a given disease with respect to the healthcare provider if they enter the community (e.g., to work or live). That is, the transmission predictions may indicate the probability that the provider will contract the disease. In some embodiments, the transmission prediction system generates an open-ended prediction (e.g., indicating the probability that the provider will contract the disease at some point). In some embodiments, the transmission prediction system generates closed predictions for one or more time periods, such as corresponding to the planned length of a work assignment for the provider (e.g., indicating the probability that the provider will contract the disease within the time period(s)).

In some embodiments, to predict the probability of transmission, the transmission prediction system predicts the future transmission rates for the community (e.g., based on historical rates and/or community vaccination rates), and predicts the probability with respect to the specific provider based on the provider's characteristics (e.g., vaccination status) and the predicted future transmission rates in the community.

As additional examples, the transmission prediction system may predict outcomes such as the probable severity of the disease if contracted by the provider, the amount of time that will elapse before the provider is able to return to work, and the like.

In some embodiments, as discussed above, the transmission prediction system may generate each transmission prediction using a corresponding machine learning model (e.g., a first model to predict probability of transmission, a second to predict probable severity, and the like). In some embodiments, the transmission prediction system may generate one or more of the transmission predictions using a single model.

In some embodiments, at block 615, the transmission prediction system generates transmission prediction(s) for the target community. In some embodiments, the transmission prediction system may also generate transmission prediction(s) for one or more other communities. For example, in addition the target community, the transmission prediction system may predict transmission probability if the provider moves to or works in one or more other communities. In some embodiments, the transmission prediction system only generates such predictions for other communities if the transmission predictions for the target community satisfy (or fail to satisfy) one or more criteria. For example, if the predicted probability of contracting the disease exceeds a threshold, the transmission prediction system may identify one or more alternative communities for the provider and generate corresponding predictions. In some embodiments, the transmission prediction system may similarly identify one or more alternative providers for the community and generate corresponding predictions.

At block 620, the transmission prediction system determines the current configuration(s) of the facility (or facilities) in the target community (e.g., the facility to which the healthcare provider may transfer or begin working in). For example, the transmission prediction system may determine the caregiver-to-patient ratio, the total number of healthcare providers and/or patients in the facility, the preventative or treatment protocols in place (e.g., whether facemasks are required, suggested, or not used, whether hand sanitizer is available in each room), and the like. In some aspects, as discussed above, one or more of such factors may additionally or alternative be used as model input. In other embodiments, one or more of such factors may be implicitly used as model input (e.g., by identifying the target community or facility) without being explicitly indicated.

At block 625, the transmission prediction system generates one or more modifications to the determined facility configuration(s). As discussed above, the configuration modifications can generally include any change or modification to the facility and/or provider that mitigate or reduce the predicted probability of transmission and/or the predicted severity. For example, the modifications may include changing prevention protocols, administering updated vaccinations, and the like. In some embodiments, generating the configuration modifications comprises suggesting or indicating the modifications (e.g., via a display). In some embodiments, the transmission prediction system facilitates implementation of the modifications (e.g., by providing them to an individual who is able to act on them, by indicating how they can be implemented, and the like). In some embodiments, the transmission prediction system may itself actually implement one or more of the modifications (e.g., by adjusting staffing, instructing providers in the facility to begin using a given protocol, and the like).

In some embodiments, the transmission prediction system generates configuration modifications based on defined rules or mappings. For example, the transmission prediction system may determine a set of potential preventative measures, identify any measures on the list that are not currently implemented by the facility, and suggest such measures be implemented. In some embodiments, the transmission prediction system may generate new transmission prediction(s) based on alternative configurations in order generate the configuration modifications. For example, the transmission prediction system may modify the community characteristics and/or provider characteristics (e.g., to indicate higher vaccination rates, to indicate use of one or more prevention protocols, and the like), and generate a second set of transmission predictions using these modified data. By identifying combination(s) of modifications that result in the most-improved transmission predictions, the transmission prediction system can generate proposed configuration modifications that best respond to the predicted future events.

In these ways, the transmission prediction system is able to use objective evaluation (rather than subjective analysis) and accurate future predictions to provide a wide variety of improvements, including improved healthcare outcomes (e.g., reduced disease transmission and/or severity, as compared to conventional systems), reduced resource usage (e.g., because fewer individuals contract the disease), more efficient allocation of resources that are used, and the like. Further, by generating facility modifications and/or alternative predictions only under certain circumstances (e.g., only when the initial predictions for the target community meet certain criteria), the transmission prediction system can reduce the computational expense of evaluating the data and enabling accurate and reliable decision making.

In some embodiments, in addition to generating various reconfiguration, the transmission prediction system may perform one or more additional actions to facilitate treatment of the patient(s) and/or care provider(s). As an example, in some embodiments, based on the predicted outcome(s), the referral prediction system may initiate one or more preventative or proactive actions. For example, if the prediction(s) indicate that a given healthcare provider may develop or contract a disease, the transmission prediction system may initiate one or more proactive measures, such as notifying the user(s) to watch closely for symptoms of the disease. As another example, the transmission prediction system may ensure that adequate supplies and training are available in the event that the disease occurs. For example, the transmission prediction system may evaluate the current inventory of treatment supplies, evaluate the current expertise of the caregiver(s), and assign specific caregiver(s) based on their expertise with respect to the predicted disease(s). As another example, the transmission prediction system may assign training or instruction sessions for the caregiver(s) to ensure that they are up-to-date with respect to detecting and/or treating the predicted disease(s).

In some embodiments, therefore, the transmission prediction system can facilitate or provide improved treatment to patients and care providers in the facilities. For example, these proactive treatment operations can reduce negative outcomes, such as by ensuring that the predicted diseases are identified quickly (e.g., such that care providers can identify contraction earlier than they otherwise would have, because they are specifically watching closely for such symptoms), and/or that patients (and providers) are treated more readily (e.g., because the diseases are caught sooner and the caregivers are better trained with supplies more readily available), and the like.

### Example Method for Determining Relevant Characteristics Data for Predicting Disease Transmission

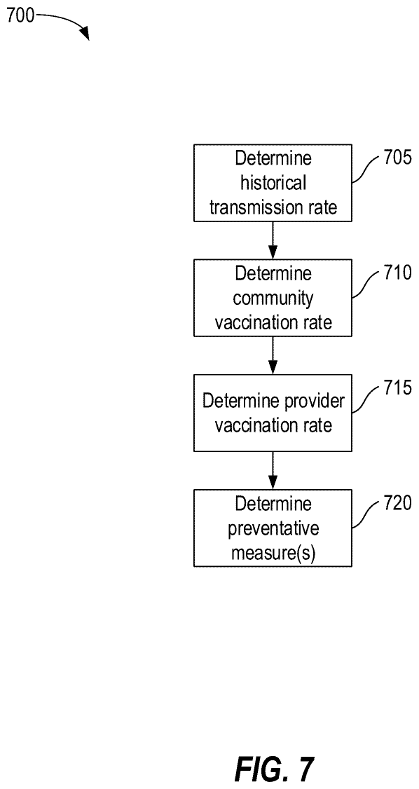

FIG. 7 is a flow diagram depicting an example method 700 for determining relevant characteristics data for predicting disease transmission, according to one embodiment of the present disclosure. In some embodiments, the method 700 is performed by a transmission prediction system, such as the transmission prediction system 125 of FIG. 1. In some embodiments, the method 700 provides additional detail for blocks 605 and/or 610 of FIG. 6.

At block 705, the transmission prediction system determines a historical transmission rate of one or more communicable diseases with respect to a target community. For example, as discussed above, the transmission prediction system may determine the historical transmission data 220 of FIG. 2, the historical transmission data 320 of FIG. 3, and/or the historical transmission data 420 of FIG. 4. In some embodiments, the particular disease(s) that the transmission prediction system evaluates may be selected or indicated by an individual or entity that initiated the prediction process. For example, if the provider and/or other individual are concerned about one or more specific diseases, the user may instruct the transmission prediction system to generate predicts with respect to these specific diseases. In some embodiments, if multiple variants or strains of a given disease exist, the transmission prediction system may evaluate which variant(s) are present in the community, and/or the prevalence or transmission rates of each such variant.

At block 710, the transmission prediction system determines community vaccination information for the target community with respect to one or more communicable diseases. For example, as discussed above, the transmission prediction system may evaluate the percentage of individuals in the community that are vaccinated, community proportions related to the type or recency of such vaccinations, and the like. As one example, the transmission prediction system may determine the community vaccination status 215 of FIG. 2, the community vaccination status 315 of FIG. 3, and/or the community vaccination status 415 of FIG. 4).

Although not depicted in the illustrated example, in some embodiments, the transmission prediction system may additionally determine one or more other community-specific characteristics or attributes, such as the total population, the population density, and the like.

At block 715, the transmission prediction system determines vaccination information for the target healthcare provider with respect to one or more communicable diseases. For example, as discussed above, the transmission prediction system may determine whether the provider has been vaccinated, whether the vaccinations they received are current, what type(s) of vaccinations the provider received, how recently the provider received the vaccination(s), and the like. As one example, the transmission prediction system may determine the provider vaccination status 210 of FIG. 2, the provider vaccination status 310 of FIG. 3, and/or the provider vaccination status 410 of FIG. 4.

At block 720, the transmission prediction system determines any preventative measures or protocols in use in the facility and/or community, and/or by the provider. For example, as discussed above, the transmission prediction system may determine whether social distancing is being practiced (e.g., the percentage of individuals in the community who indicate that they are social distancing), whether masks are required, preferred, discouraged, or otherwise in use (e.g., the percentage of individuals in the community and/or facility who indicate they wear a mask), what testing protocols are in place (e.g., testing in response to symptom development, testing periodically regardless of symptom development, and the like), whether hand sanitizer is readily available, and the like. Generally, the particular preventative measures that may be evaluated may vary depending on the particular implementation, as well as based on the particular disease(s) that are currently spreading in the community (and how such diseases are spread).

### Example Method for Generating Staffing, Supply, and/or Protocol Modifications Based on Disease Predictions Using Machine Learning

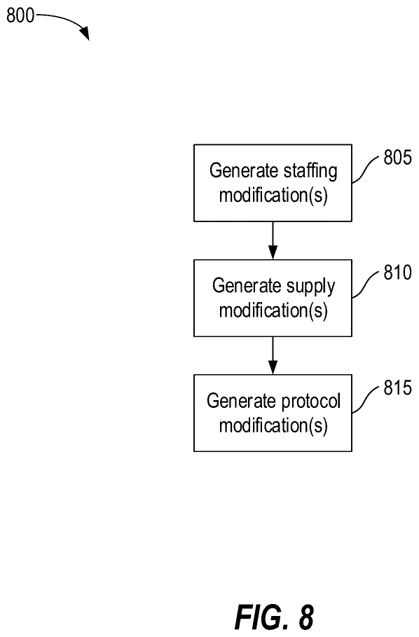

FIG. 8 is a flow diagram depicting an example method 800 for generating staffing, supply, and/or protocol modifications based on disease predictions using machine learning, according to one embodiment of the present disclosure. In some embodiments, the method 800 is performed by a transmission prediction system, such as the transmission prediction system 125 of FIG. 1. In some embodiments, the method 800 provides additional detail for block 625 of FIG. 6.

Generally, each block in the method 800 may be an optional step, and the transmission prediction system may use some, all, or none of the depicted examples when generating configuration modifications. Additionally, at each block in the method 800, generating a modification may generally comprise selecting, evaluating, suggesting, facilitating implementation of, and/or actually implementing the modification. For example, the transmission prediction system may select the modification from a set of possible alternative modifications, evaluate the modification to predict its efficacy, suggest the modification (e.g., based on determining that the modification is predicted to be helpful) to an individual with authority to implement the modification, and/or actually implement the modification (e.g., by reconfiguring one or more computing systems).

At block 805, the transmission prediction system generates one or more staffing modifications for the target facility. For example, as discussed above, the transmission prediction system may suggest that one or more additional healthcare providers also be assigned to the target facility, that one or more providers who are currently assigned to the facility be reassigned to a different facility, that the mix or ratio of staff members having different skillsets or training be adjusted, and the like. In one aspect, for example, the transmission prediction system may assign the target healthcare provider (for whom the transmission predictions are being generated) to the target facility. For example, the transmission prediction system may determine that the predicted transmission is below a threshold, and therefore determine to assign the provider to the facility. As another example, the transmission prediction system may determine that the predicted transmission is above a threshold, and therefore determine to refrain from assigning the provider (or to assign the provider in conjunction with one or more other actions, such as supply or protocol reconfigurations, or other staffing changes).

At block 810, the transmission prediction system generates one or more supply modifications for the target facility. For example, as discussed above, the transmission prediction system may suggest that one or more additional supplies be provided to the facility (e.g., increasing the supply of sanitizer, gloves or other personal protected equipment (PPE), treatment equipment such as ventilators, and the like). As one example, if the transmission prediction system determines that the predicted transmission is sufficiently high and/or the predicted future transmission rates at the facility are high, the transmission prediction system may determine that such additional supplies are warranted. As another example, if the transmission prediction system determines that the transmission probability is low, the transmission prediction system may determine that additional supplies are not needed. In some aspects, the supply modification ma include reducing the supplies allotted to the facility in the future. In some embodiments, the supply configuration is determined based at least in part on protocol modifications, as discussed below.

At block 815, the transmission prediction system generates one or more protocol modifications for the target facility. For example, as discussed above, the transmission prediction system may suggest that testing be increased (e.g., testing weekly instead of monthly), that additional or different PPE be used (e.g., instructing each healthcare provider in the facility to wear a different type of glove), that protocols such as social distancing be used or additional sanitation, and the like. In some embodiments, as discussed above, the protocol modifications may be coupled with supply modifications as needed (e.g., to provide additional PPE for the facility).

In these ways, the transmission prediction system may generate a wide variety of suggested modifications based on the transmission predictions, enabling substantially improved outcomes for patients as well as more efficient allocations of resources.

### Example Method for Generating Updated Configurations Based on Disease Predictions Using Machine Learning

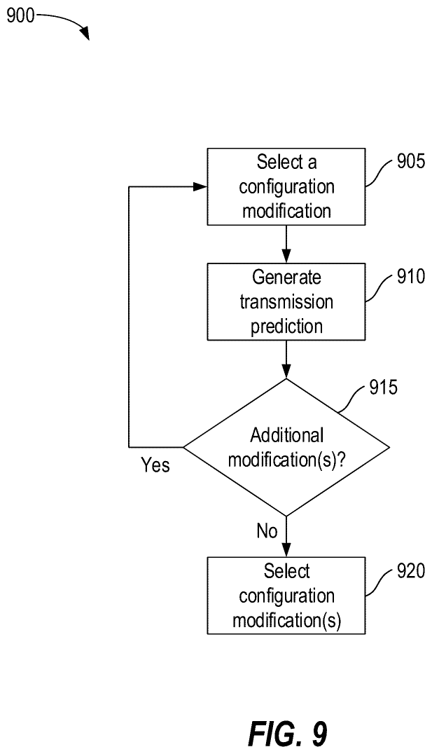

FIG. 9 is a flow diagram depicting an example method 900 for generating updated configurations based on disease predictions using machine learning, according to one embodiment of the present disclosure. In some embodiments, the method 900 is performed by a transmission prediction system, such as the transmission prediction system 125 of FIG. 1. In some embodiments, the method 900 provides additional detail for block 625 of FIG. 6, and/or for blocks 805, 810, and 815 of FIG. 8.

At block 905, the transmission prediction system selects a configuration modification for evaluation. In some embodiments, the transmission prediction system selects the modification from a set or list of potential or alternative modifications. For example, when evaluating staffing modifications, the transmission prediction system may select a healthcare provider that can be potentially assigned to the target facility, and/or may select a healthcare provider (currently working at the target facility) that may be potentially reassigned to a different facility. As another example, when evaluating alternative prevention protocols, the transmission prediction system may select a particular protocol (or combination of protocols) from a defined list.

In some embodiments, the set of potential modifications can generally include any change to any mutable characteristic of a healthcare facility, a healthcare provider, and/or a surrounding community, as discussed above. In an embodiment, the transmission prediction system may use any suitable technique or criteria to select the configuration modification (including randomly or pseudo-randomly), as each alternative may be evaluated during the method 900.

At block 910, the transmission prediction system generates one or more transmission predictions based on the selected configuration modification. In some embodiments, as discussed above, the transmission prediction system generates the transmission predictions using machine learning. For example, the transmission prediction system may determine the initial inputs (e.g., a combination of a healthcare provider and a target facility), and modify these inputs based on the selected modification (e.g., to indicate that different preventative measures are in use at the facility, to indicate different vaccination status of the provider, and the like). These modified inputs can then be used to generate a transmission prediction that simulates or represents what may happen if the change is implemented.

In some embodiments, at block 910, the transmission prediction system generates one or more transmission predictions based on selection or input from a user. For example, if the user wishes to reduce the probability of disease transmission to the healthcare provider, the user may specify this goal and the transmission prediction system may generate transmission predictions corresponding to this outcome (refraining from generating predictions relating to other outcomes). This may substantially reduce the computational expense of using the machine learning models.

At block 915, the transmission prediction system determines whether there is at least one additional (potential) modification that has not-yet been evaluated. If so, the method 900 returns to block 905. If not, the method 900 continues to block 920. Although the illustrated example depicts a sequential process (evaluating each alternative in turn) for conceptual clarity, in some embodiments, the transmission prediction system may select and evaluate some or all of the modifications in parallel.

At block 920, the transmission prediction system selects one or more configuration modifications based on the generated transmission predictions. For example, in some embodiments, the transmission prediction system identifies the modification(s) that resulted in the best transmission predictions based on various criteria, such as the modification that resulted in the lowest probability of disease transmission, the lowest predicted future transmission rate in a facility or community, the lowest predicted disease severity, and the like. In some embodiments, rather than selecting one or more of the best modifications, the transmission prediction system may output any modifications that resulted in a positive change (e.g., that at least reduced the probability of transmission, even if the probability was only reduced slightly).

In some embodiments, selecting the modification(s) includes facilitating implementation of the modification(s), such as by suggesting them to a user or directly causing their implementation. In some embodiments, the transmission prediction system outputs the selected modifications and allows the user to select or approve one or more modifications for implementation. For example, as discussed above, the transmission prediction system may suggest that a different healthcare provider be assigned to the target facility, that the target healthcare provider be assigned to a different facility, that one or more preventative actions be taken, such as increased testing and/or vaccination incentives, and the like.

In these ways, the transmission prediction system can use machine learning to evaluate alternative modifications in such a way as to substantially improve the resulting outcomes while simultaneously reducing computational expense and improving the general operations of the transmission prediction system and other systems.

### Example Method for Selecting Alternative Providers Based on Disease Transmission Using Machine Learning

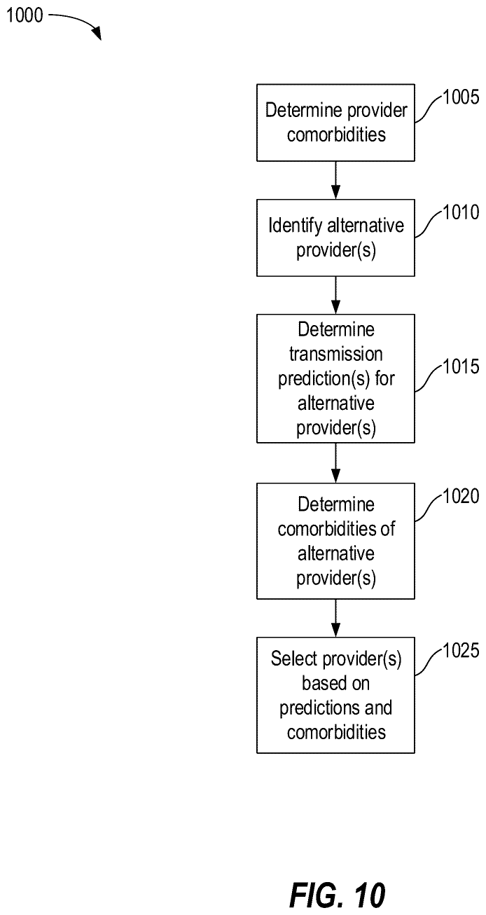

FIG. 10 is a flow diagram depicting an example method 1000 for selecting alternative providers based on disease transmission using machine learning, according to one embodiment of the present disclosure. In some embodiments, the method 1000 is performed by a transmission prediction system, such as the transmission prediction system 125 of FIG. 1. In some embodiments, the transmission prediction system provides additional detail for block 805 of FIG. 8.

At block 1005, the transmission prediction system determines one or more comorbidities of a target healthcare provider with respect to one or more communicable diseases. Generally, as discussed above, the particular comorbidities of interest may vary depending on the particular disease, and may include diagnoses or conditions such as obesity, high blood pressure, diabetes, and the like. In some embodiments, based on such comorbidities, the transmission prediction system may predict the potential severity of the disease (if the provider contracts the disease), as discussed above. In some embodiments, as discussed above, the predicted disease severity may be combined with the predicted probability of transmission for the provider to generate a unified or aggregated prediction (e.g., weighting the predicted severity based on the probability of transmission). In some embodiments, the transmission prediction system determines to evaluate alternative providers in response to determining that the target provider (with one or more comorbidities) has a predicted probability of transmission that meets or exceeds a minimum threshold (e.g., because the predicted severity is high and the probability of transmission is sufficiently high to warrant evaluation of alternatives).

At block 1010, the transmission prediction system identifies one or more alternative providers for the target facility. That is, the transmission prediction system identifies any other healthcare providers that may alternatively be assigned to the given facility, in addition to or instead of assigning the target provider to the facility.

At block 1015, the transmission prediction system determines or generates one or more transmission predictions for each alternative provider. For example, as discussed above, the transmission prediction system may process the provider-specific information (e.g., vaccination status of each) using one or more machine learning models to predict the probability that the respective provider will contract a disease if they are assigned to the facility, the probability that they will experience severe disease if they contract the disease, and the like.

At block 1020, the transmission prediction system determines any comorbidities of the alternative healthcare providers. For example, as discussed above, the transmission prediction system may determine whether any of the providers have more, fewer, or different comorbidities as compared to the target provider.

At block 1025, the transmission prediction system then selects one or more healthcare providers to be assigned to the target facility based on the transmission predictions (e.g., probabilities of transmission) and determined comorbidities (e.g., predicted disease severity). For example, if the probabilities of transmission are comparable (e.g., within a threshold difference) for the target provider and an alternative provider, but the alternative provider has substantially fewer comorbidities, the transmission prediction system may determine to assign the alternative provider to the facility. Similarly, in some embodiments, the transmission prediction system may assign an alternative provider with lower comorbidities (e.g., reduced predicted severity), even if the probability of transmission is substantially higher.

In these ways, the transmission prediction system can minimize (or at least reduce) the predicted cumulative impact of communicable diseases. For example, the transmission prediction system can assign staff that reduces the probability of transmission, that reduces the probability of severe infection, and/or that balances the probabilities of transmission and severe disease. This reduces burden on the individual users as well as on the healthcare system and community as a whole.

### Example Method for Refining Machine Learning Models Based on Disease Transmission Feedback

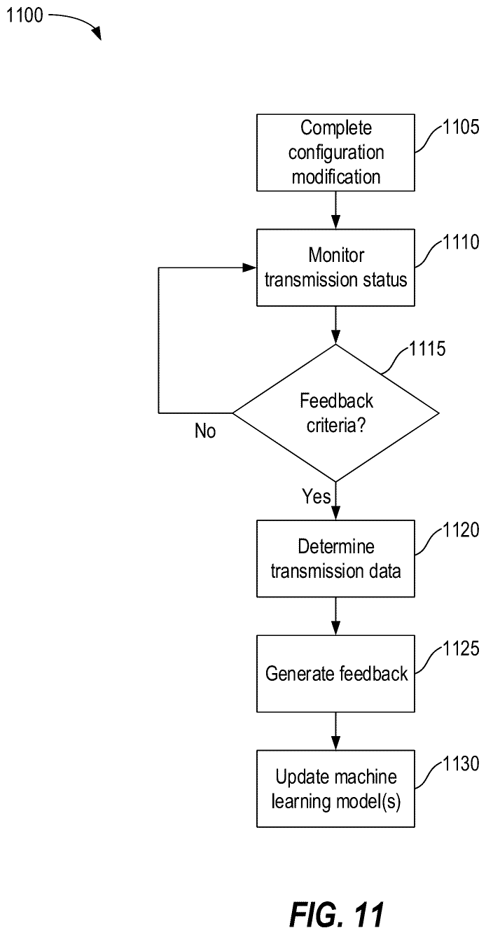

FIG. 11 is a flow diagram depicting an example method 1100 for refining machine learning models based on disease transmission feedback, according to one embodiment of the present disclosure. In some embodiments, the method 1100 is performed by a transmission prediction system, such as the transmission prediction system 125 of FIG. 1. In some embodiments, the method 1100 provides additional detail for the workflow 400 of FIG. 4.

At block 1105, the transmission prediction system completes a configuration modification. For example, as discussed above, completing the modification may include evaluating it using machine learning (as discussed above) to generate transmission prediction(s). In some aspects, completing the configuration modification includes facilitating acceptance or declination of the modification. For example, if the modification is accepted (e.g., by a user), the modification may be implemented (e.g., staff assignments may be finalized).

At block 1110, the transmission prediction system monitors the transmission status of the healthcare provider that was assigned to or works in the selected facility or community. For example, the transmission prediction system may periodically determine whether the provider has contracted one or more communicable diseases, the severity of any diseases that the provider has contracted, and the like. In some embodiments, the transmission prediction system may receive these updates using push operations from other systems. In other embodiments, the transmission prediction system may request or retrieve these updates using pull operations.

At block 1115, the transmission prediction system determines whether one or more feedback criteria are met. For example, based on the monitoring of the provider status, the transmission prediction system may determine whether feedback related to transmission outcomes is available to refine the machine learning model(s) (e.g., if the provider has contracted the disease, if a sufficient amount of time has passed without the provider contracting the disease, and the like). If not, the method 1100 returns to block 1110.

If, at block 1115, the transmission prediction system determines that feedback is available, the method 1100 continues to block 1120, where the transmission prediction system determines the updated transmission data for the provider, facility, and/or community. For example, as discussed above, the transmission prediction system may determine whether the provider contracted the disease, whether transmission rates in the community and/or facility increased, decreased, or remained the same, the severity of any diseases that the provider contracted, and the like. In some aspects, the updated transmission data corresponds to the transmission data 445 of FIG. 4.

At block 1125, the transmission prediction system generates feedback based on the updated data. For example, the transmission prediction system may preprocess the transmission data or otherwise format it in such a way that it can be used to train the model(s). For example, the transmission prediction system may use the transmission data to generate one or more labels, and associate the label(s) with the corresponding input data (e.g., provider status, community vaccination rates, and the like)) from the time when the configuration modification was first evaluated or completed. In this way, the transmission prediction system can dynamically generate new training exemplars whenever new outcome data is available.

At block 1130, the transmission prediction system updates the machine learning model(s) based on the newly generated feedback. For example, as discussed above, the transmission prediction system may update one or more facility-specific or community-specific models (for the facility or community where the provider is working), one or more disease-specific models (for the disease(s) the provider contracted), and/or one or more outcome-specific models (e.g., based on the severity of the disease for the provider).

As discussed above, the particular operations used to refine the models may vary depending on the particular implementation. For example, in the case of a neural network architecture, the transmission prediction system may compare the actual outcome to the (original) predicted outcome to compute a loss, and use backpropagation to update the models based on the loss.

Although the illustrated example depicts updating of the model(s) using a single set of new feedback (e.g., using stochastic gradient descent), in some embodiments, the transmission prediction system may instead store the feedback until a later time, and update the model(s) based on batches of samples (e.g., using batch gradient descent).

In these ways, the transmission prediction system may continuously or periodically update the machine learning models during inferencing, enabling generation of highly accurate predictions.

### Example Method for Training Machine Learning Models to Predict Disease Transmission

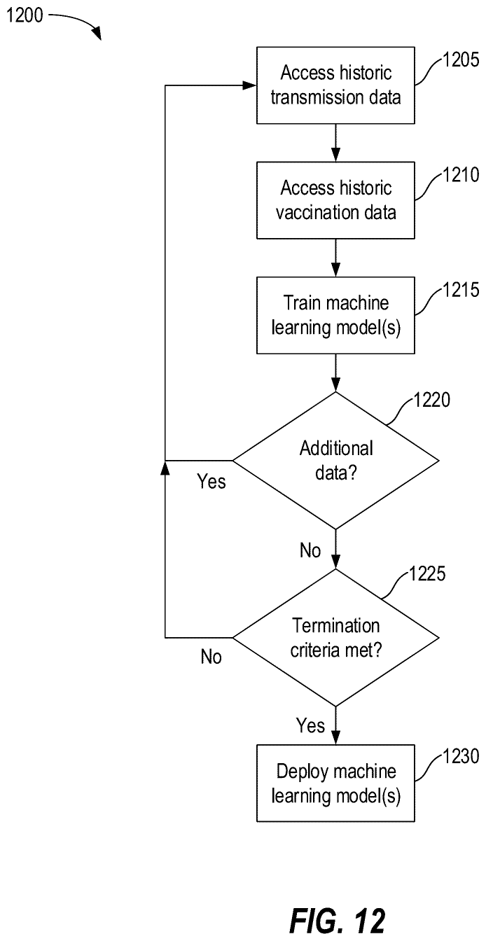

FIG. 12 is a flow diagram depicting an example method 1200 for training machine learning models to predict disease transmission, according to one embodiment of the present disclosure. In some embodiments, the method 1200 is performed by a transmission prediction system, such as the transmission prediction system 125 of FIG. 1. In some embodiments, the method 1200 is performed by a machine learning system, such as the machine learning system 515 of FIG. 5.

At block 1205, the transmission prediction system accesses a set of historic transmission data. As discussed above, the historical transmission data generally includes information for one or more healthcare facilities at one or more prior points in time. For example, the historical transmission data may include information such as the transmission rate of one or more communicable diseases at one or more points in time. In some embodiments, the historic transmission data may correspond to the transmission data 510 of FIG. 5.

At block 1210, the transmission prediction system accesses historic vaccination data corresponding to the set of historic transmission data. That is, the transmission prediction system may access records of vaccinations for the community that corresponds to the transmission data, for the healthcare facility, for providers in the facility, and the like. As discussed above, the vaccination data can generally include information such as whether a given provider had been vaccinated, the vaccination rate in the community, the type(s) of vaccines that had been administered, and the like. In some embodiments, the historic vaccination data may correspond to the vaccination data 513 of FIG. 5.

At block 1215, the transmission prediction system trains one or more machine learning models based on the historic transmission data and vaccination data. As discussed above, the particular operations used to train the models may vary depending on the particular implementation. For example, in the case of a neural network architecture, the transmission prediction system may compare the actual transmission outcomes for one or more providers to a predicted transmission outcome (generated by processing the transmission data and/or vaccination data using the model) to compute a loss, and use backpropagation to update the models based on the loss.

Although the illustrated example depicts updating of the model(s) using a single set of historic data (e.g., using stochastic gradient descent for each individual provider or record), in some embodiments, the transmission prediction system may instead update the model(s) based on batches of data (e.g., using batch gradient descent).

In some embodiments, at block 1220, the transmission prediction system may train one or more facility-specific and/or community-specific models (e.g., models trained specifically to generate predictions for a given healthcare facility or community). In some embodiments, the transmission prediction system may train one or more facility-agnostic models (e.g., models trained to generate predictions for any facility or community). In some embodiments, the transmission prediction system may train one or more disease-specific models (e.g., models trained to generate predictions for the specific disease(s) of interest). In some embodiments, the transmission prediction system may train one or more outcome-specific models (e.g., models trained to generate predictions for the specific outcome(s), such as probability of transmission, disease severity, and the like).

At block 1220, the transmission prediction system determines whether there are any additional historic records in the historic transmission data that have not yet been used to train the model(s). If so, the method 1200 returns to block 1205 to select or access another record. Although the illustrated example depicts an iterative process (selecting and evaluating each historic record in turn) for conceptual clarity, in some embodiments, the transmission prediction system may train the models based on some or all of the data in parallel.

Returning to block 1220, if the transmission prediction system determines that all available historic data has been used for training in the current iteration, the method 1200 continues to block 1225, where the transmission prediction system determines whether one or more termination criteria are met. The termination criteria may generally include a wide variety of evaluations depending on the particular implementation. For example, in some embodiments, the transmission prediction system may determine whether a defined number of iterations or epochs have been completed. In some embodiments, the transmission prediction system determines whether a desired or preferred prediction accuracy has been reached. In some embodiments, the transmission prediction system may determine whether a defined amount of resources have been spent training the model. In some embodiments, the transmission prediction system determines whether any new records are ready for training.

If, at block 1225, the transmission prediction system determines that the termination criteria are not met, the method 1200 returns to block 1205 to access a new (or the same) set of historic data. If the transmission prediction system determines that the termination criteria are met, the method 1200 continues to block 1230, where the transmission prediction system deploys the trained machine learning model(s) for inferencing.

As discussed above, deploying the model(s) may generally include any operations needed to prepare or provide the models for generating runtime inferences, such as transmitting the model parameter(s) to inferencing system(s), instantiating the model locally, and the like.

### Example Method for Using Machine Learning to Predict Transmission

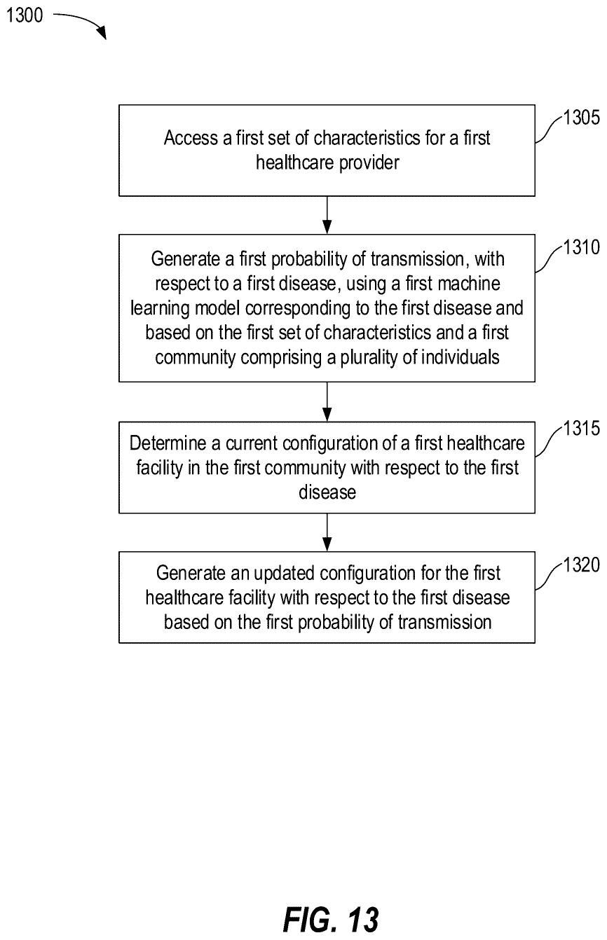

FIG. 13 is a flow diagram depicting an example method 1300 for using machine learning to predict transmission, according to one embodiment of the present disclosure. In some embodiments, the method 1300 is performed by a transmission prediction system, such as the transmission prediction system 125 of FIG. 1. In some embodiments, the method 1300 is performed by a machine learning system, such as the machine learning system 515 of FIG. 5.

At block 1305, a first set of characteristics (e.g., provider data 110 of FIG. 1) for a first healthcare provider (e.g., provider 105 of FIG. 1) is accessed.

At block 1310, a first probability of transmission (e.g., a transmission prediction 130 of FIG. 1), with respect to a first disease, is generated using a first machine learning model (e.g., machine learning model 230 of FIGS. 2-4) corresponding to the first disease and based on the first set of characteristics and a first community (e.g., for a healthcare facility 120 of FIG. 1) comprising a plurality of individuals.

At block 1315, a current configuration of a first healthcare facility in the first community with respect to the first disease is determined.

At block 1320, an updated configuration (e.g., updated configuration 340 of FIG. 3) is generated for the first healthcare facility with respect to the first disease based on the first probability of transmission.

### Example Method for Training Machine Learning Models to Predict Transmission

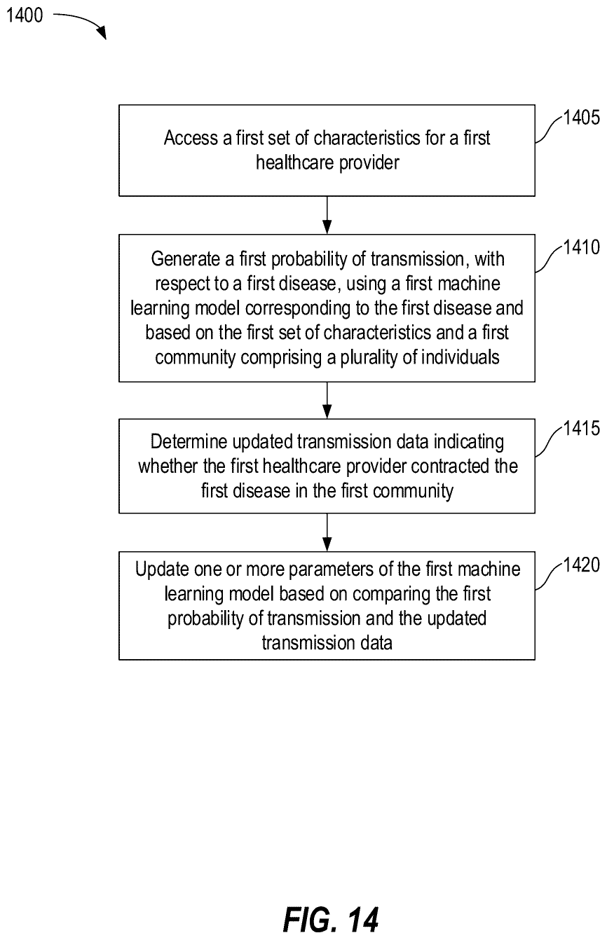

FIG. 14 is a flow diagram depicting an example method 1400 for training machine learning models to predict transmission, according to one embodiment of the present disclosure. In some embodiments, the method 1400 is performed by a transmission prediction system, such as the transmission prediction system 125 of FIG. 1.

At block 1405, a first set of characteristics (e.g., provider data 110 of FIG. 1) for a first healthcare provider (e.g., provider 105 of FIG. 1) is accessed.

At block 1410, a first probability of transmission (e.g., a transmission prediction 130 of FIG. 1), with respect to a first disease, is generated using a first machine learning model (e.g., machine learning model 230 of FIGS. 2-4) corresponding to the first disease and based on the first set of characteristics and a first community (e.g., for a healthcare facility 120 of FIG. 1) comprising a plurality of individuals.

At block 1415, updated transmission data (e.g., transmission data 445 of FIG. 4) indicating whether the first healthcare provider contracted the first disease in the first community is determined.

At block 1420, one or more parameters of the first machine learning model are updated based on comparing the first probability of transmission and the updated transmission data.

### Example Computing Device for Predicting Transmission Using Machine Learning

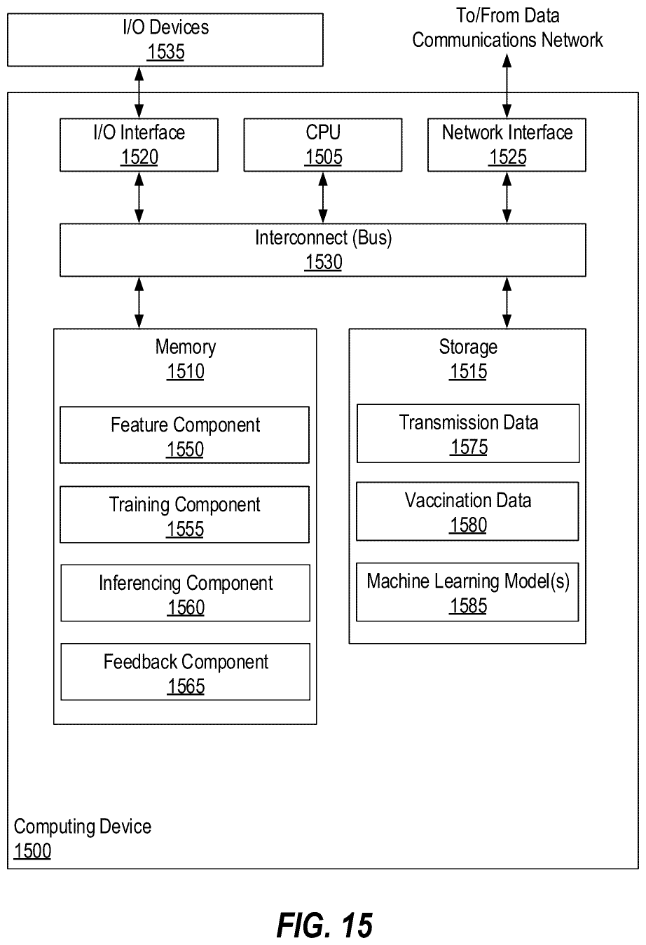

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In some embodiments, the computing device 1500 corresponds to or implements a transmission prediction system, such as the transmission prediction system 125 of FIG. 1. In some embodiments, the computing device 1500 corresponds to or implements a feedback system, such as the feedback component 440 of FIG. 4. In some embodiments, the computing device 1500 corresponds to or implements a machine learning system, such as the machine learning system 515 of FIG. 5. Generally, the computing device 1500 may correspond to or implement any system that trains, updates, and/or uses machine learning models to predict disease transmission outcomes based on prior transmission and vaccination data.

As illustrated, the computing device 1500 includes a CPU 1505, memory 1510, a network interface 1525, and one or more I/O interfaces 1520. Though not included in the depicted example, in some embodiments, the computing device 1500 also includes one or more storages. In the illustrated embodiment, the CPU 1505 retrieves and executes programming instructions stored in memory 1510, as well as stores and retrieves application data residing in memory 1510 and/or storage (not depicted). The CPU 1505 is generally representative of a single CPU and/or GPU, multiple CPUs and/or GPUs, a single CPU and/or GPU having multiple processing cores, and the like. The memory 1510 is generally included to be representative of a random access memory. In an embodiment, if storage is present, it may include any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, network interface(s) 1525, and I/O interface(s) 1520 are communicatively coupled by one or more buses 1530.

In the illustrated embodiment, the memory 1510 includes a feature component 1550, a training component 1555, an inferencing component 1560, and a feedback component 1565, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

The feature component 1550 may generally be used to identify, extract, and/or generate feature data from transmission and/or vaccination data, as discussed above. For example, the feature component 1550 may evaluate provider data and/or community data (e.g., provider data 110 and/or community data 115 of FIG. 1, input data 205 of FIG. 2, input data 305 of FIG. 3, input data 405 of FIG. 4, and/or training data 505 of FIG. 5) to generate model input (e.g., a feature tensor) that encodes this information for more efficient machine learning.

The training component 1555 may be used to train one or more machine learning models (such as machine learning models 1585) based on historic transmission and vaccination data, as discussed above. The inferencing component 1560 may be used to generate transmission predictions by processing provider and/or community data using one or more machine learning models (such as machine learning models 1585), as discussed above. The feedback component 1565 may be used to collect and/or generate feedback based on transmission outcomes, allowing the feedback component 1565 and/or the training component 1555 to update the machine learning models continuously or periodically.

In the illustrated example, the storage 1515 includes transmission data 1575, vaccination data 1580, and machine learning models 1585. Although depicted as residing in storage 1515, the depicted data may be stored in any suitable location. In at least one embodiment, as discussed above, the transmission data 1575, vaccination data 1580, and machine learning models 1585 may be stored in separate repositories.

Generally, the transmission data 1575 may include disease transmission information for one or more diseases, as discussed above. For example, the transmission data 1575 may correspond to training data, such as the training data 505 of FIG. 5, and may include information such as the transmission data 510 of FIG. 5. In some embodiments, the training component 1555 uses the transmission data 1575 to train machine learning models 1585, as discussed above.

The vaccination data 1580 (which may correspond to the vaccination data 513 of FIG. 5) may include information relating to vaccination of individuals (e.g., specific care providers) and/or communities (e.g., the vaccination rate in the community). The machine learning model(s) 1585 may generally correspond to one or more models trained to generate transmission predictions based on input provider and/or community data, such as the machine learning models 230 of FIGS. 2-5.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., the components of the transmission prediction system 125 of FIG. 1) or related data available in the cloud. For example, the transmission prediction system 125 could execute on a computing system in the cloud and generate transmission predictions. In such a case, the transmission prediction system 125 could store and execute machine learning models in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a first set of characteristics for a first healthcare provider;
generating a first probability of transmission, with respect to a first disease, using a first machine learning model corresponding to the first disease and based on the first set of characteristics and a first community comprising a plurality of individuals;
determining a current configuration of a first healthcare facility in the first community with respect to the first disease; and
generating an updated configuration for the first healthcare facility with respect to the first disease based on the first probability of transmission.

2. The method of claim 1, further comprising:
determining a vaccination rate of the plurality of individuals with respect to the first disease; and
determining a historical transmission rate of the first community with respect to the first disease.

3. The method of claim 1, wherein the first machine learning model was trained based on historical transmission data for the first disease.

4. The method of claim 1, wherein:
the current configuration comprises a set of healthcare providers staffing the first healthcare facility, and
generating the updated configuration comprises adding the first healthcare provider to the set of healthcare providers based on the first probability of transmission.

5. The method of claim 1, wherein:
the current configuration comprises a set of healthcare providers staffing the first healthcare facility, and
generating the updated configuration comprises adding a second healthcare provider to the set of healthcare providers based on the first probability of transmission.

6. The method of claim 5, further comprising:
accessing a second set of characteristics for the second healthcare provider;
generating a second probability of transmission for the second healthcare provider with respect to the first disease using the first machine learning model; and
determining that the second probability of transmission is lower than the first probability of transmission.

7. The method of claim 5, further comprising:
determining that the first probability of transmission satisfies a minimum threshold;
determining a first set of one or more comorbidities of the first healthcare provider with respect to the first disease;
determining a second set of one or more comorbidities of the second healthcare provider with respect to the first disease; and
determining to add the second healthcare provider to the set of healthcare providers based on the first and second sets of comorbidities.

8. The method of claim 5, further comprising:
generating a second probability of transmission for the first healthcare provider using the first machine learning model based on the first set of characteristics and a second community; and
generating an updated configuration for a second healthcare facility in the second community comprising adding the first healthcare provider to a set of healthcare providers staffing the second healthcare facility.

9. The method of claim 1, wherein:
the current configuration comprises a set of supplies allocated to the first healthcare facility with respect to the first disease, and
generating the updated configuration comprises adding one or more additional supplies to the set of allocated supplies based on the first probability of transmission.

10. The method of claim 1, wherein:
the current configuration comprises a set of protocols used by the first healthcare facility with respect to the first disease, and
generating the updated configuration comprises adding one or more additional protocols to the set of protocols based on the first probability of transmission.

11. The method of claim 1, wherein the first set of characteristics comprise an indication as to whether the first healthcare provider is vaccinated against the first disease.

12. The method of claim 1, further comprising:
generating a second probability of transmission for the first healthcare provider with respect to a second disease using a second machine learning model corresponding to the second disease; and
generating an updated configuration for the first healthcare facility with respect to the second disease based on the second probability of transmission.

13. A method, comprising:
accessing a first set of characteristics for a first healthcare provider;
generating a first probability of transmission, with respect to a first disease, using a first machine learning model corresponding to the first disease and based on the first set of characteristics and a first community comprising a plurality of individuals;
determining updated transmission data indicating whether the first healthcare provider contracted the first disease in the first community; and
updating one or more parameters of the first machine learning model based on comparing the first probability of transmission and the updated transmission data.

14. The method of claim 13, the operation further comprising:
determining a vaccination rate of the plurality of individuals with respect to the first disease; and
determining a historical transmission rate of the first community with respect to the first disease.

15. The method of claim 13, wherein the first set of characteristics comprise an indication as to whether the first healthcare provider is vaccinated against the first disease.

16. One or more non-transitory computer-readable media comprising computer-executable instructions that, when executed by one or more processors of one or more processing systems, cause the one or more processing systems to perform an operation comprising:
accessing a first set of characteristics for a first healthcare provider;
generating a first probability of transmission, with respect to a first disease, using a first machine learning model corresponding to the first disease and based on the first set of characteristics and a first community comprising a plurality of individuals;
determining a current configuration of a first healthcare facility in the first community with respect to the first disease; and
generating an updated configuration for the first healthcare facility with respect to the first disease based on the first probability of transmission.

17. The non-transitory computer-readable media of claim 16, the operation further comprising:
determining a vaccination rate of the plurality of individuals with respect to the first disease; and
determining a historical transmission rate of the first community with respect to the first disease.

18. The non-transitory computer-readable media of claim 16, wherein:
the current configuration comprises a set of healthcare providers staffing the first healthcare facility, and
generating the updated configuration comprises adding the first healthcare provider to the set of healthcare providers based on the first probability of transmission.

19. The non-transitory computer-readable media of claim 16, wherein:
the current configuration comprises a set of healthcare providers staffing the first healthcare facility, and
generating the updated configuration comprises adding a second healthcare provider to the set of healthcare providers based on the first probability of transmission.

20. The non-transitory computer-readable media of claim 19, the operation further comprising:
accessing a second set of characteristics for the second healthcare provider;
generating a second probability of transmission for the second healthcare provider with respect to the first disease using the first machine learning model; and
determining that the second probability of transmission is lower than the first probability of transmission.
